SEQUENCE LISTING (30 sequences)

SEQ ID NO: 1 - protein, 22 aa, Homo sapiens
AALAAFNAQNNGSNFQLEEISR

SEQ ID NO: 2 - protein, 20 aa, Homo sapiens
AFITNFSMIIDGMTYPGIIK

SEQ ID NO: 3 - protein, 16 aa, Homo sapiens
  MOD_RES (14)..(14): Cysteine carbamidomethyl
ALPQPQNVTSLLGCTH

SEQ ID NO: 4 - protein, 15 aa, Homo sapiens
DLQSLEDILHQVENK

SEQ ID NO: 5 - protein, 18 aa, Homo sapiens
  MOD_RES (3)..(3): Cysteine carbamidomethyl
DQCIVDDITYNVNDTFHK

SEQ ID NO: 6 - protein, 16 aa, Homo sapiens
EHEGAIYPDNTTDFQR

SEQ ID NO: 7 - protein, 17 aa, Homo sapiens
ELHHLQEQNVSNAFLDK

SEQ ID NO: 8 - protein, 9 aa, Homo sapiens
  MOD_RES (3)..(3): Cysteine carbamidomethyl
FGCEIENNR

SEQ ID NO: 9 - protein, 20 aa, Homo sapiens
FNLTETSEAEIHQSFQHLLR

SEQ ID NO: 10 - protein, 25 aa, Homo sapiens
  MOD_RES (2)..(2): Cysteine carbamidomethyl
ICDLLVANNHFAHFFAPQNLTNMNK

SEQ ID NO: 11 - protein, 13 aa, Homo sapiens
  MOD_RES (9)..(9): Cysteine carbamidomethyl
ISEENETTCYMGK

SEQ ID NO: 12 - protein, 14 aa, Homo sapiens
LANLTQGEDQYYLR

SEQ ID NO: 13 - protein, 20 aa, Homo sapiens
LDAPTNLQFVNETDSTVLVR

SEQ ID NO: 14 - protein, 16 aa, Homo sapiens
  MOD_RES (4)..(4): Cysteine carbamidomethyl
LGACNDTLQQLMEVFK

SEQ ID NO: 15 - protein, 11 aa, Homo sapiens
  MOD_RES (10)..(10): Cysteine carbamidomethyl
LGNWSAMPSCK

SEQ ID NO: 16 - protein, 25 aa, Homo sapiens
LPTQNITFQTESSVAEQEAEFQSPK

SEQ ID NO: 17 - protein, 19 aa, Homo sapiens
  MOD_RES (15)..(15): Cysteine carbamidomethyl
LQAPLNYTEFQKPICLPSK

SEQ ID NO: 18 - protein, 13 aa, Homo sapiens
NLSMPLLPADFHK

SEQ ID NO: 19 - protein, 33 aa, Homo sapiens
  MOD_RES (9)..(9): Cysteine carbamidomethyl
QVFPGLNYCTSGAYSNASSTDSASYYPLTGDTR

SEQ ID NO: 20 - protein, 19 aa, Homo sapiens
SLTFNETYQDISELVYGAK

SEQ ID NO: 21 - protein, 13 aa, Homo sapiens
  MOD_RES (8)..(8): Cysteine carbamidomethyl
SWPAVGNCSSALR

SEQ ID NO: 22 - protein, 15 aa, Homo sapiens
  MOD_RES (2)..(2): Cysteine carbamidomethyl
  MOD_RES (5)..(5): Cysteine carbamidomethyl
VCQDCPLLAPLNDTR

SEQ ID NO: 23 - protein, 18 aa, Homo sapiens
VDKDLQSLEDILHQVENK

SEQ ID NO: 24 - protein, 21 aa, Homo sapiens
VGQLQLSHNLSLVILVPQNLK

SEQ ID NO: 25 - protein, 19 aa, Homo sapiens
VSNQTLSLFFTVLQDVPVR

SEQ ID NO: 26 - protein, 11 aa, Homo sapiens
LGNWSAMPSCK

SEQ ID NO: 27 - protein, 31 aa, Homo sapiens
QVFPGLNYCTSGAYSNASSTDSASYYPLTGD

SEQ ID NO: 28 - protein, 13 aa, Homo sapiens
SWPAVGNCSSALR

SEQ ID NO: 29 - protein, 15 aa, Homo sapiens
VCQDCPLLAPLNDTR

SEQ ID NO: 30 - protein, 9 aa, Homo sapiens
FGCEIENNR

(19) United States
(12) Patent Application Publication
(10) Pub. No.: US 2023/0213533 A1
(43) Pub. Date: Jul. 6, 2023

(54) SITE- AND STRUCTURE-SPECIFIC CORE FUCOSYLATION IN LIVER DISEASE

(71) Applicant: Georgetown University, Washington, DC (US)

(72) Inventors: Radoslav GOLDMAN, Reston, VA (US); Miloslav SANDA, Arlington, VA (US)

(21) Appl. No.: 17/749,029

(22) Filed: May 19, 2022

Related U.S. Application Data
(60) Provisional application No. 63/191,573, filed on May 21, 2021.

(51) Int. Cl. G01N 33/68

(52) U.S. Cl. CPC G01N 33/6893; G01N 33/6848; G01N 2800/085

(57) ABSTRACT

The instant disclosure provides methods of detecting N-glycopeptides in a sample by contacting the sample with one or more exoglycosidases and detecting the N-glycopeptides by mass spectrometry. Also provided are methods of detecting the presence or progression of a liver disease and treating said liver disease.

### CROSS-REFERENCE TO RELATED APPLICATIONS

This application claims the benefit of U.S. Provisional Application No. 63/191,573, filed May 21, 2021, the disclosure of which is incorporated herein by reference.

### STATEMENT REGARDING FEDERALLY SPONSORED RESEARCH OR DEVELOPMENT

This invention was made with government support under U01CA230692, R01CA135069, R01CA238455, and S100D023557 awarded by the National Institutes of Health. The United States Government has certain rights in the invention.

### SEQUENCE LISTING

The instant application contains a Sequence Listing which has been submitted electronically in ASCII format and is hereby incorporated by reference in its entirety. Said ASCII copy, created on Jul. 13, 2022, is named 729648_GUS-032_SL.txt and is 7,641 bytes in size.

### BACKGROUND

Carbohydrates form one of the major groups of biological macromolecules in living organisms. Many biological processes including protein folding, stability, immune response, and receptor activation are regulated by glycosylation. Fucosylation of proteins regulates such processes and is associated with various diseases including autoimmunity and cancer. Mass spectrometry efficiently identifies structures of fucosylated glycans or sites of core fucosylated N-glycopeptides but quantification of the glycopeptides remains difficult and less explored. Accordingly, there exists a need in the art for improved methods of detecting N-glycopeptides and their numerous glycoforms.

### BRIEF SUMMARY OF THE INVENTION

In one aspect, the disclosure provides a method of detecting N-glycopeptides in a sample, the method comprising: a) contacting the sample with one or more exoglycosidases, thereby producing an exoglycosidase-treated sample; and b) detecting the N-glycopeptides in the exoglycosidase-treated sample by mass spectrometry.

In certain embodiments, the exoglycosidase is a neuraminidase or a fucosidase.

In certain embodiments, the neuraminidase is α2-3,6,8,9 neuraminidase.

In certain embodiments, the fucosidase is α1-2 fucosidase or α1-3,4 fucosidase.

In certain embodiments, the sample is contacted with at least one neuraminidase followed by at least one fucosidase.

In certain embodiments, the sample is contacted with α2-3,6,8,9 neuraminidase followed by α1-2 fucosidase or α1-3,4 fucosidase.

In certain embodiments, the sample is contacted with α2-3,6,8,9 neuraminidase followed by α1-2 fucosidase and α1-3,4 fucosidase.

In certain embodiments, the sample is contacted with α2-3,6,8,9 neuraminidase at about 37° C. for about 8 to about 24 hours followed by α1-2 fucosidase and α1-3,4 fucosidase at about 37° C. for about 8 to about 24 hours.

In certain embodiments, the sample is not contacted with an endoglycosidase.

In certain embodiments, the sample is contacted with an affinity reagent prior to contacting with the one or more exoglycosidases to remove a substantial portion of abundant proteins.

In certain embodiments, the abundant proteins comprise one or more of albumin, IgG, antitrypsin, IgA, transferrin, haptoglobin, fibrinogen, alpha2-macroglobulin, alpha1-acid glycoprotein, IgM, apolipoprotein AI, apolipoprotein AII, complement C3, and transthyretin.

In certain embodiments, at least about 90% of the abundant proteins are removed.

In certain embodiments, the sample is contacted with trypsin prior to contacting with the one or more exoglycosidases.

In certain embodiments, the mass spectrometry comprises liquid chromatography - tandem mass spectrometry (LC-MS-MS).

In certain embodiments, the LC-MS-MS comprises data-independent acquisition (DIA) LC-MS-MS.

In certain embodiments, the mass spectrometry is performed with low collision energy (CE) fragmentation, optionally wherein the low CE fragmentation comprises less than about 50%, less than about 40%, less than about 30%, or less than about 10% normalized collision energy (NCE).

In certain embodiments, the mass spectrometry is performed with an MS1 scan of a resolution of about 30,000 to about 120,000 at about 400 to about 2000 m/z.

In certain embodiments, the mass spectrometry is performed with an MS2 scan of a resolution of about 7,500 to about 30,000 at about 100 to about 2000 m/z.

In certain embodiments, the mass spectrometry detects core fucosylation of the N-glycopeptides.

In certain embodiments, the mass spectrometry detects bi-, tri-, and tetra-antennary forms of a specific N-glycopeptide.

In certain embodiments, the sample comprises serum, urine, or cerebrospinal fluid (CSF). In certain embodiments, the sample is isolated from a subject.

In certain embodiments, the subject is suffering from a liver disease or suspected of having a liver disease. In certain embodiments, the of liver disease is liver cirrhosis. In certain embodiments, the liver cirrhosis is of an alcoholic (ALD), a hepatitis B viral (HBV), a hepatitis C viral (HCV), and/or a non-alcoholic steatohepatitis (NASH) etiology.

In another aspect, the disclosure provides a method of detecting the presence or progression of a liver disease in a subject, the method comprising: a) contacting a sample isolated from the subject with one or more exoglycosidases, thereby producing an exoglycosidase-treated sample; and b) detecting in the exoglycosidase-treated sample by mass spectrometry one or both of: i-a) core fucosylation of N-glycopeptides, and ii-a) bi-, tri-, and tetra-antennary forms of a specific N-glycopeptide, wherein: i-b) an increase of core fucosylation relative to a control sample indicates the presence or progression of the liver disease in the subject, and ii-b1) a decrease of the bi-antennary form of the specific N-glycopeptide relative to a control sample indicates the presence or progression of liver disease in the subject, or ii-b2) an increase of the tri- and/or tetra-antennary form of the specific N-glycopeptide relative to a control sample indicates the presence or progression of liver disease in the subject.

In certain embodiments, an increase in the ratio of fucosylated bi-, tri-, or tetra-antennary forms of a specific N-glycopeptide to non-fucosylated bi-, tri-, or tetra-antennary forms of a specific N-glycopeptide relative to a control sample indicates the presence or progression of liver disease in the subject.

In certain embodiments, the increase of core fucosylation relative to a control sample is at least about 1.5-fold (i.e., about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold).

In certain embodiments, the exoglycosidase is a neuraminidase or a fucosidase.

In certain embodiments, the neuraminidase is α2-3,6,8,9 neuraminidase.

In certain embodiments, the fucosidase is α1-2 fucosidase or α1-3,4 fucosidase.

In certain embodiments, the sample is contacted with at least one neuraminidase followed by at least one fucosidase. In certain embodiments, the sample is contacted with α2-3,6,8,9 neuraminidase followed by α1-2 fucosidase or α1-3,4 fucosidase. In certain embodiments, the sample is contacted with α2-3,6,8,9 neuraminidase followed by α1-2 fucosidase and α1-3,4 fucosidase. In certain embodiments, the sample is contacted with α2-3,6,8,9 neuraminidase at about 37° C. for about 8 to about 24 hours followed by α1-2 fucosidase and α1-3,4 fucosidase at about 37° C. for about 8 to about 24 hours.

In certain embodiments, the sample is not contacted with an endoglycosidase.

In certain embodiments, the sample is contacted with an affinity reagent prior to contacting with the one or more exoglycosidases to remove a substantial portion of abundant proteins.

In certain embodiments, the abundant proteins comprise one or more of albumin, IgG, antitrypsin, IgA, transferrin, haptoglobin, fibrinogen, alpha2-macroglobulin, alpha1-acid glycoprotein, IgM, apolipoprotein AI, apolipoprotein AII, complement C3, and transthyretin.

In certain embodiments, at least about 90% of the abundant proteins are removed.

In certain embodiments, the sample is contacted with trypsin prior to contacting with the one or more exoglycosidases.

In certain embodiments, the mass spectrometry comprises liquid chromatography - tandem mass spectrometry (LC-MS-MS). In certain embodiments, the LC-MS-MS comprises data-independent acquisition (DIA) LC-MS-MS.

In certain embodiments, the mass spectrometry is performed with low collision energy (CE) fragmentation, optionally wherein the low CE fragmentation comprises less than about 50%, less than about 40%, less than about 30%, or less than about 10% normalized collision energy (NCE).

In certain embodiments, the mass spectrometry is performed with an MS1 scan of a resolution of about 30,000 to about 120,000 at about 400 to about 2000 m/z.

In certain embodiments, the mass spectrometry is performed with an MS2 scan of a resolution of about 7,500 to about 30,000 at about 100 to about 2000 m/z.

In certain embodiments, the sample comprises serum.

In certain embodiments, the of liver disease is liver cirrhosis.

In certain embodiments, the liver cirrhosis is of an alcoholic (ALD), a hepatitis B viral (HBV), a hepatitis C viral (HCV), and/or a non-alcoholic steatohepatitis (NASH) etiology.

In certain embodiments, the N-glycopeptide comprises one or more of: AALAAFNAQNNGSNFQLEEISR (SEQ ID NO: 1); AFITNFSMIIDGMTYPGIIK (SEQ ID NO: 2); ALPQPQNVTSLLGC(cam)TH (SEQ ID NO: 3); DLQSLEDILHQVENK (SEQ ID NO: 4); DQC(cam)IVDDITYNVNDTFHK (SEQ ID NO: 5); EHEGAIYPDNTTDFQR (SEQ ID NO: 6); ELHHLQEQNVSNAFLDK (SEQ ID NO: 7); FGC(cam)EIENNR (SEQ ID NO: 8); FNLTETSEAEIHQSFQHLLR (SEQ ID NO: 9); IC(cam)DLLVANNHFAHFFAPQNLTNMNK (SEQ ID NO: 10); ISEENETTC(cam)YMGK (SEQ ID NO: 11); LANLTQGEDQYYLR (SEQ ID NO: 12); LDAPTNLQFVNETDSTVLVR (SEQ ID NO: 13); LGAC(cam)NDTLQQLMEVFK (SEQ ID NO: 14); LGNWSAMPSC(cam)K (SEQ ID NO: 15); LPTQNITFQTESSVAEQEAEFQSPK (SEQ ID NO: 16); LQAPLNYTEFQKPIC(cam)LPSK (SEQ ID NO: 17); NLSMPLLPADFHK (SEQ ID NO: 18); QVFPGLNYC(cam)TSGAYSNASSTDSASYYPLTGDTR (SEQ ID NO: 19); SLTFNETYQDISELVYGAK (SEQ ID NO: 20); SWPAVGNC(cam)SSALR (SEQ ID NO: 21); VC(cam)QDC(cam)PLLAPLNDTR (SEQ ID NO: 22); VDKDLQSLEDILHQVENK (SEQ ID NO: 23); VGQLQLSHNLSLVILVPQNLK (SEQ ID NO: 24); VSNQTLSLFFTVLQDVPVR (SEQ ID NO: 25); LGNWSAMPSCK (SEQ ID NO: 26); and QVFPGLNYCTSGAYSNASSTDSASYYPLTGD (SEQ ID NO: 27).

In certain embodiments, the N-glycopeptide comprises one or more of: ELHHLQEQNVSNAFLDK (SEQ ID NO: 7); LANLTQGEDQYYLR (SEQ ID NO: 12); LGNWSAMPSCK (SEQ ID NO: 26); LDAPTNLQFVNETDSTVLVR (SEQ ID NO: 13); LPTQNITFQTESSVAEQEAEFQSPK (SEQ ID NO: 16); QVFPGLNYCTSGAYSNASSTDSASYYPLTGD (SEQ ID NO: 27); and VDKDLQSLEDILHQVENK(SEQ ID NO: 23).

In another aspect, the disclosure provides a method of treating a liver disease in a subject, the method comprising: a) contacting a sample isolated from the subject with one or more exoglycosidases, thereby producing an exoglycosidase-treated sample; b) detecting in the exoglycosidase-treated sample by mass spectrometry one or both of: i-a) core fucosylation of N-glycopeptides, and ii-a) bi-, tri-, and tetra-antennary forms of a specific N-glycopeptide; and c) administering a therapy to the subject to treat the liver disease, wherein the therapy is administered if: i-b) an increase of core fucosylation relative to a control sample is detected in the subject, ii-b1) a decrease of the bi-antennary form of the specific N-glycopeptide relative to a control sample is detected in the subject, and/or ii-b2) an increase of the tri- and/or tetra-antennary form of the specific N-glycopeptide relative to a control sample is detected in the subject.

In certain embodiments, the therapy is useful to treat the liver disease.

In certain embodiments, the of liver disease is liver cirrhosis.

In certain embodiments, the liver cirrhosis is of an alcoholic (ALD), a hepatitis B viral (HBV), a hepatitis C viral (HCV), and/or a non-alcoholic steatohepatitis (NASH) etiology.

In certain embodiments, the therapy comprises a liver cirrhosis therapy.

In certain embodiments, the liver cirrhosis therapy comprises one or more of naltrexone, acamprosate, vitamin E, pioglitazone, an antiviral drug, an interferon, and ursodiol.

In certain embodiments, the antiviral drug is selected from the group consisting of entecavir, tenofovir, lamivudine, adefovir, and telbivudine.

In certain embodiments, the interferon comprises interferon alpha 2b or pegylated interferon.

In certain embodiments, the N-glycopeptide comprises one or more of: AALAAFNAQNNGSNFQLEEISR (SEQ ID NO: 1); AFITNFSMIIDGMTYPGIIK (SEQ ID NO: 2); ALPQPQNVTSLLGC(cam)TH (SEQ ID NO: 3); DLQSLEDILHQVENK (SEQ ID NO: 4); DQC(cam)IVDDITYNVNDTFHK (SEQ ID NO: 5); EHEGAIYPDNTTDFQR (SEQ ID NO: 6); ELHHLQEQNVSNAFLDK (SEQ ID NO: 7); FGC(cam)EIENNR (SEQ ID NO: 8); FNLTETSEAEIHQSFQHLLR (SEQ ID NO: 9); IC(cam)DLLVANNHFAHFFAPQNLTNMNK (SEQ ID NO: 10); ISEENETTC(cam)YMGK (SEQ ID NO: 11); LANLTQGEDQYYLR (SEQ ID NO: 12); LDAPTNLQFVNETDSTVLVR (SEQ ID NO: 13); LGAC(cam)NDTLQQLMEVFK (SEQ ID NO: 14); LGNWSAMPSC(cam)K (SEQ ID NO: 15); LPTQNITFQTESSVAEQEAEFQSPK (SEQ ID NO: 16); LQAPLNYTEFQKPIC(cam)LPSK (SEQ ID NO: 17); NLSMPLLPADFHK (SEQ ID NO: 18); QVFPGLNYC(cam)TSGAYSNASSTDSASYYPLTGDTR (SEQ ID NO: 19); SLTFNETYQDISELVYGAK (SEQ ID NO: 20); SWPAVGNC(cam)SSALR (SEQ ID NO: 21); VC(cam)QDC(cam)PLLAPLNDTR (SEQ ID NO: 22); VDKDLQSLEDILHQVENK (SEQ ID NO: 23); VGQLQLSHNLSLVILVPQNLK (SEQ ID NO: 24); VSNQTLSLFFTVLQDVPVR (SEQ ID NO: 25); LGNWSAMPSCK (SEQ ID NO: 26); and QVFPGLNYCTSGAYSNASSTDSASYYPLTGD (SEQ ID NO: 27).

In certain embodiments, the N-glycopeptide comprises one or more of: ELHHLQEQNVSNAFLDK (SEQ ID NO: 7); LANLTQGEDQYYLR (SEQ ID NO: 12); LGNWSAMPSCK (SEQ ID NO: 26); LDAPTNLQFVNETDSTVLVR (SEQ ID NO: 13); LPTQNITFQTESSVAEQEAEFQSPK (SEQ ID NO: 16); QVFPGLNYCTSGAYSNASSTDSASYYPLTGD (SEQ II) NO: 27); and VDKDLQSLEDILHQVENK (SEQ ID NO: 23).

### BRIEF DESCRIPTION OF THE FIGURES

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1A - FIG. 1B show low collision energy spectra from the GP-SWATH DIA of the following glycoforms of the SWPAVGNCSSALR (SEQ ID NO: 28) glycopeptides of hemopexin: bi-antennary core fucosylated glycoform (FIG. 1A); and bi-antennary glycoform without fucose (FIG. 1B).

FIG. 2 shows a XIC chromatogram of six glycoforms of the SWPAVGNCSSALR (SEQ ID NO: 28) glycopeptide of hemopexin extracted from the GP-SWATH DIA data of a MARS 14 depleted serum sample treated with α2-3,6,8,9 neuraminidase, α1-2 and α1-3,4 fucosidases.

FIG. 3A - FIG. 3B show changes in the relative abundance of the bi-antennary (FIG. 3A) and tri-antennary core (FIG. 3B) fucosylated glycoforms of the LANLTQGEDQYYLR (SEQ ID NO: 12) peptide of clusterin in healthy controls and patients with liver cirrhosis of the indicated etiologies.

FIG. 4A- FIG. 4B show the ratio of the increase in the intensity of the following core fucosylated glycopeptides in the liver cirrhosis groups of four etiologies compared to the healthy controls: bi-, tri- and tetra-antennary glycoforms of the VCQDCPLLAPLNDTR (SEQ ID NO: 29) peptide of Alpha-2-HS-glycoprotein (FIG. 4A); bi-, tri-, and tetra-antennary glycoforms of the LGNWSAMPSCK (SEQ ID NO: 26) peptide of beta-2- glycoprotein (FIG. 4B).

FIG. 5 is a table showing the distribution of the average coefficients of variability (CV) for the indicated glycopeptide classes and disease groups across the entire study set. Duplicate measurements were performed for both the (A) technical and (B) biological CV measures.

FIG. 6 shows HILIC chromatograms of the glycopeptide of hemopexin treated with fucosidase. These results confirm cleavage of the outer-arm fucose with >98% efficiency. FIG. 6 discloses SEQ ID NO: 28.

### DETAILED DESCRIPTION

The instant disclosure provides a combination of exoglycosidase digests with data independent (DIA) LC-MS/MS using low collision energy (CE) fragmentation to achieve quantification of the core fucosylated glycopeptides with partially resolved N-glycan structures. This method is able to provide relative quantification of the changes in core fucosylation in disease states, such as liver diseases. In particular, the instant method identified 45 glycopeptides derived from 18 serum proteins of patients with liver cirrhosis of various etiologies, demonstrating applicability to real-world patient samples. This disclosure provides a useful platform for the diagnosis of disease, as well as monitoring disease progression, which aides in the treatment of said diseases.

As used herein, the term “data-independent acquisition mass spectrometry” (“DIA-MS”) refers to a method of molecular structure determination in which all ions within a selected m/z range are fragmented and analyzed in a second stage of tandem mass spectrometry. Tandem mass spectra are acquired either by fragmenting all ions that enter the mass spectrometer at a given time (called broadband DIA) or by sequentially isolating and fragmenting ranges of m/z. DIA is an alternative to data-dependent acquisition (DDA) where a fixed number of precursor ions are selected and analyzed by tandem mass spectrometry. DIA seeks to determine what is present in a sample of potentially unknown identity. To determine the molecular structure of sample molecules, a mass spectrometer is first used to mass analyze all sample ions (precursor ions) within a selected window of mass to charge ratio (m/z). Such a scan is often denoted as an MS1 scan. The selected sample ions are then fragmented and the resulting fragments are subsequently mass analyzed across the selected m/z range. The scan of the fragmented ions is often denoted as an MS2 scan.

In certain embodiments, the mass spectrometry is performed with an MS1 scan of a resolution of about 30,000 to about 120,000 at about 400 to about 2000 m/z. In certain embodiments, the resolution is about 30,000, about 40,000, about 50,000, about 60,000, about 70,000, about 80,000, about 90,000, about 100,000, about 110,000, or about 120,000. In certain embodiments, the m/z value is about 400, about 500, about 600, about 700, about 800, about 900, about 1000, about 1100, about 1200, about 1300, about 1400, about 1500, about 1600, about 1700, about 1800, about 1900, or about 2000 m/z.

In certain embodiments, the mass spectrometry is performed with an MS2 scan of a resolution of about 7,500 to about 30,000 at about 100 to about 2000 m/z. In certain embodiments, the resolution is about 7,500, about 10,000, about 15,000, about 20,000, about 25,000, or about 30,000. In certain embodiments, the m/z value is about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 m/z.

One approach to DIA data analysis is based on a targeted analysis, also known as SWATH-MS (Sequential Windowed Acquisition of All Theoretical Fragment Ion Mass Spectra). This approach uses targeted extraction of fragment ion traces directly for identification and quantification without an explicit attempt to de-multiplex the DIA fragment ion spectra.

As used herein, the term “collision energy” (“CE”) refers to the energy used to fragment analytes in the mass spectrometer. “Low CE fragmentation” refers to a reduced level of energy relative to a normalized value. In certain embodiments, the low CE fragmentation comprises less than about 50%, less than about 40%, less than about 30%, or less than about 10% normalized collision energy (NCE).

As used herein, the term “core fucosylation” or “core fucose” refers to fucose that is attached by α-(1-6) linkage to the GlcNAc bound to a protein’s asparagine residue.

As used herein, the term “antenna fucosylation” or “antenna fucose” refers to outer-arm or terminal fucosylation, that is attached to the antennae of the complex type N-glycans by α-(1-3) and α-(1-4) linkage to the GlcNAc residues, by α-(1-2) linkage to galactose, or by α-(1-2) linkage to GalNAc.

All N-linked glycans are based on the common core pentasaccharide, Man₃GlcNAc₂. GlcNAc sequences added to the N-linked glycan core are called “antennae”. A “bi-antennary” glycan has two GlcNAc branches linked to the core. A “tri-antennary” glycan has three GlcNAc branches. A “tetra-antennary” glycan has four GlcNAc branches.

As used herein, the term “N-glycopeptide” refers to a peptide that is N-glycosylated. The N-glycopeptide may be detectable in a sample obtained from a subject (e.g., a subject suffering from a liver disease). A specific N-glycopeptide, defined by a specific amino acid sequence, may come in numerous glycoforms, each glycoform having a unique glycan structure. A specific N-glycopeptide may have a bi-antennary glycoform, a tri-antennary glycoform, or a tetra-antennary glycoform.

As used herein, the term “exoglycosidase” refers to a glycoside hydrolase enzyme that cleaves the glycosidic linkage of a terminal monosaccharide in an oligosaccharide or polysaccharide. Non-limiting examples of exoglycosidases include neuraminidases, fucosidases, mannosidases, and galactosidases. In certain embodiments, the exoglycosidase is selected from the group consisting of: α1-2 Fucosidase, α1-2,3 Mannosidase, α1-2,3,4,6 Fucosidase, α1-2,3,6 Mannosidase, α1-2,4,6 Fucosidase O, α1-3,4 Fucosidase, α1-3,4,6 Galactosidase, α1-3,6 Galactosidase, α1-6 Mannosidase, α2-3 Neuraminidase S, α2-3,6,8 Neuraminidase, α2-3,6,8,9 Neuraminidase A, α-N-Acetylgalactosaminidase, β1-3 Galactosidase, β1-3,4 Galactosidase, β1-4 Galactosidase S, β-N-Acetylglucosaminidase S, and β-N-Acetylhexosaminidase f.

In certain embodiments, the neuraminidase is α2-3,6,8,9 neuraminidase.

In certain embodiments, the fucosidase is α1-2 fucosidase or α1-3,4 fucosidase.

As used herein, the term “endoglycosidase” refers to a glycoside hydrolase enzyme that cleaves the glycosidic linkage of a non-terminal monosaccharide in an oligosaccharide or polysaccharide (i.e., cleaving polysaccharide chains between residues that are not the terminal residue).

### EXAMPLES

The following examples are put forth so as to provide those of ordinary skill in the art with a complete disclosure and description of how to make and use the methods and compositions featured in the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Experimental Set-Up

### Materials and Reagents

Acetonitrile (LC-MS grade), water (LC-MS grade), and dithiothreitol (DTT) were obtained from ThermoFisher Scientific (Waltham, MA). Iodoacetamide (IAA) was from MP Biomedicals (Santa Ana, CA). Trypsin Gold (V5280) was obtained from Promega (Madison, WI). α2-3,6,8,9 neuraminidase (P0722), α1-2 (P0724) and α1-3,4 (P0769) fucosidases with GlycoBuffer 1 (5 mM calcium chloride, 50 mM sodium acetate, pH 5.5) were purchased from New England BioLabs (Ipswich, MA). Hemopexin from human plasma was supplied by Athens Research and Technology (Athens, Georgia). Rapigest SF was from Waters (Milford, MA). All other chemicals were obtained from SigmaAldrich (Saint Louis, MO).

### Study Population

Applicability of the method was documented on serum samples of healthy volunteers and patients with cirrhosis of the liver of hepatitis C (HCV), hepatitis B (HBV), alcoholic (ALD) and non-alcoholic steatohepatitis (NASH) etiologies as described previously.³⁴ Briefly, participants were enrolled in collaboration with the Department of Hepatology and Liver Transplantation, Georgetown University Hospital, Washington, DC, under protocols approved by the Institutional Review Board. Blood samples were collected using red-top serum vacutainer tubes (BD Diagnostics, Franklin Lakes, NJ) in line with manufacturer’s recommendations and samples were processed within 6 h of the blood draw for storage at -80° C. Two pooled samples were created for each disease category. Each pool represents five participants and duplicate samples were analyzed of each of the pools (4 samples total per group). All the pools were age-matched and had a comparable degree of liver damage, as measured by the model for end-stage liver disease (MELD) score.

### Sample Processing

Affinity depletion of abundant serum proteins was carried out on a HP1290 HPLC system (Agilent, Santa Clara, CA) using the Multiple Affinity Removal Column Human 14 (MARS 14, Agilent) according to manufacturer’s protocol. Briefly, human serum (20 µL) was mixed with buffer A (85 µL), remaining particulates were removed by centrifugation through a 0.22-µm spin filter 1 min at 16,000 × g, and the clarified serum was injected on the column. Affinity buffer A and B were used as mobile phases, 100% A at a flow rate 0.25 mL/min at 0-9 min, 100% B at a flow rate 1 mL/min at 9-15 min, and 100% A at a flow rate 1 mL/min at 15-20 min. Collected flow-thru of each sample was precipitated using 80% acetone overnight at -20° C., washed 2 times with 100% acetone, and dissolved in 50 mM ammonium bicarbonate buffer pH 8 for determination of the protein concentration by BCA assay. Aliquots of the samples (10 µg of total protein) were reduced with 5 mM DTT for 60 min at 60° C., alkylated with 15 mM IAA for 30 min in the dark, residual IAA was quenched with 5 mM DTT, and digested with 2.5 ng/µL Trypsin Gold (Promega, Madison, WI) at 37° C. in Barocycler NEP2320 (Pressure BioSciences, South Easton, MA) for 1 hour. Trypsin was deactivated by heating to 99° C. for 10 minutes, samples were evaporated using a vacuum concentrator (Labconco, Kansas City, MO) and digested with 2 µL of α2-3,6,8,9 neuraminidase in GlycoBuffer 1 overnight at 37° C. according to manufacturer’s instructions. Neuraminidase was deactivated by heating at 75° C. for 10 minutes and 2 µL aliquots of each 1-3,4 fucosidase and 1-2 fucosidase were added to the samples for an overnight digestion at 37° C. The digests were evaporated using a vacuum concentrator (Labconco) and reconstituted at a final concentration 0.5 µg/µL in a mobile phase A (0.1% formic acid in 2% acetonitrile) for mass spectrometric analysis.

### Glycopeptide Identification by Nano LC-MS/MS

Following 5 min trapping/washing step in solvent A (2% acetonitrile, 0.1% formic acid) at 10 µL/min, peptide and glycopeptide separation was achieved at a flow rate of 0.3 µL/min using the following gradient: 0-3 min 2% B (0.1% formic acid in ACN), 3-5 min 2-10% B; 5-60 min 10- 45% B; 60-65 min 45-98% B; 65-70 min 98% B, 70-90 min equilibration at 2% B. An Orbitrap Fusion Lumos mass spectrometer was used to analyze the glycopeptides with the electrospray ionization voltage at 3 kV and the capillary temperature at 275° C. MS1 scans were performed over m/z 400-1800 with the wide quadrupole isolation on a resolution of 120,000 (m/z 200), RF Lens at 40%, intensity threshold for MS2 set to 2.0e4, selected precursors for MS2 with charge state 3-8, and dynamic exclusion 30 s. Data-dependent HCD tandem mass spectra were collected with a resolution of 15,000 in the Orbitrap with fixed first mass 110 and normalized collision energy 10-30%. LC-MS datasets were processed by Protein Discoverer 2.2. (Thermo Fisher Scientific, Waltham, MA) with Byonic node (Protein Metrics, Cupertino, CA) followed by manual confirmation of the glycopeptides.

### SWATH DIA Quantification by Nano LC-MS/MS

Glycopeptide separation was achieved on a Nanoacquity LC (Waters, Milford, MA) using capillary trap, 180 µm × 0.5 mm, and analytical 75 µm × 150 Atlantis DB C18, 3 µm, 300 Å columns (Water, Milford, MA) interfaced with 6600 TripleTOF (Sciex, Framingham, MA). A 3 min trapping step using 2% ACN, 0.1% formic acid at 15 µL/min was followed by chromatographic separation at 0.4 µL/min as follows: starting conditions 5% ACN, 0.1% formic acid; 1-55 min, 5-50% ACN, 0.1% formic acid; 55-60 min, 50-95% ACN, 0.1% formic acid; 60-70 min 95% ACN, 0.1% formic acid followed by equilibration to starting conditions for additional 20 min. For all runs, we have injected 1 µL of tryptic digest on column. Data Independent Acquisition (DIA) was used with one MS1 full scan (400-2000 m/z) and n MS/MS fragmentations (800-1600), dependent on the isolation window (15 Da), with rolling collision energy-optimized as described previously.³⁵ MS/MS spectra were recorded in the range 400-2000 m/z with resolution 30,000 and mass accuracy less than 15 ppm using the following experimental parameters: declustering potential 80V, curtain gas 30, ion spray voltage 2.300 V, ion source gas1 11, interface heater 180° C., entrance potential 10 V, collision exit potential 11 V. Precursor and product ion masses and charge states together with the glycopeptide retention times (RT) used for quantification of the glycoforms are summarized in Table 2.

### Data Analysis

Y-ion isotope clusters with isolation window of 1.2 Da, extracted from the low CE SWATHMS/MS with a 15 Da step window, were used for analysis of the glycopeptide intensities. Multiquan 2.0 software was used for processing of the quantitative results. Processing methods were created for ion extraction from each SWATH window and for each glycoform. Areas of the fucosylated glycopeptides were normalized to the areas of the corresponding non-fucosylated analytes. Coefficients of variability (CV) were determined from the means and standard deviations of the measurements and were compared across the disease groups and glycoform categories using one-way analysis of variance (ANOVA). Normalized glycopeptide intensities (measured as peak areas) and their ratios were compared in the different disease groups by Kruskal-Wallis one-way ANOVA with statistical significance determined as a two-sided p<0.05. Statistical analyses were performed using R version 3.40; further data processing and graphing were carried out in Microsoft Excel and Graphpad prism.

### Results

### Glycosidase Treatment and Identification of the N-Glycopeptides in Human Serum

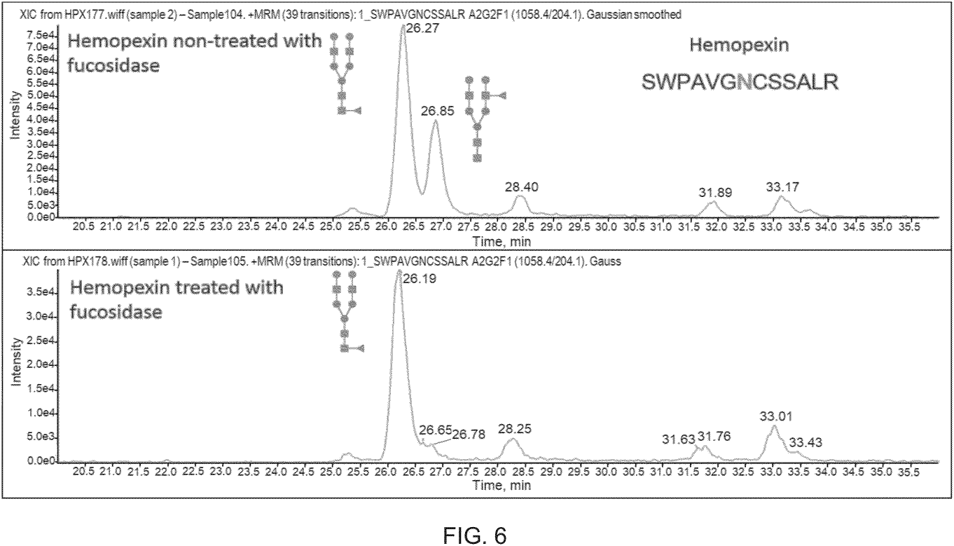

The major goal of this study was to evaluate the feasibility of DIA quantification of core fucosylated glycopeptides (GP-SWATH) without an endoglycosidase digest. This was done to evaluate whether changes in the core fucosylation of liver secreted glycoproteins observed in fibrotic liver disease differ with branching of the glycoforms. To this end, tryptic digests of MARS14 depleted human serum were analyzed. The glycopeptides were further simplified by sequential treatment with exoglycosidases (α2-3,6,8,9 neuraminidase followed by α1-2 and α1-3,4 fucosidases) and were subsequently analyzed by LC-MS/MS without any further glycopeptide enrichment. Completeness of the exoglycosidase digests were verified by nano HILIC separation of the hemopexin glycopeptide (FIG. 6) as described previously.³⁶ This assures that core fucosylated glycoforms of each peptide were quantified but allows comparison of fucosylation on glycoforms with different degree of branching.

Pooled samples were prepared of healthy controls and patients with cirrhosis of the liver caused by HCV, HBV, ALD, and NASH etiologies. For each group, two pooled samples were prepared (n=5 participants each), which were analyzed first by LC-MS/MS with HCD fragmentation on an Orbitrap Fusion Lumos as described above. For further analysis bi-, tri-, and tetra-antennary glycoforms of 45 N-glycopeptides of 18 proteins detectable in all the samples in their fucosylated and non-fucosylated forms were selected (Table 2). In addition to the 24 biantennary glycoform pairs (HexNAc4Hex5Fuc/HexNAc4Hex5), the analysis included 13 glycopeptides with tri-antennary (HexNAc5Hex6Fuc/HexNAc5Hex6) glycoforms, and ten glycopeptides with tetra-antennary (HexNAc6Hex7Fuc/HexNAc6Hex7) glycoforms. In total, 90 glycopeptides were quantified by the DIA LC-MS/MS workflow.

### Core Fucosylation and Limited Fragmentation

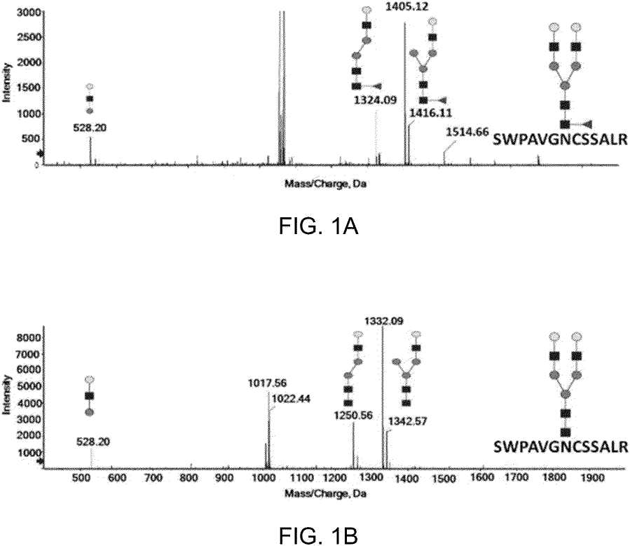

To facilitate the DIA analysis of glycopeptides, lower CE settings were used (half of the CE used for peptides) to minimize interference of the peptides, to minimize rearrangement of fucose and unwanted fragmentation of the glycans, and to maximize the yield of the quantification fragments as described previously.^{35,37} Transitions for the quantification of the 90 glycopeptides use the Y-ions corresponding, typically, to the loss of an outer-arm of the glycan; this generates a Y-ion of [m/z-1] compared to the precursor and the corresponding oxonium ion. The low CE fragmentation of the core fucosylated and non-fucosylated glycopeptide pairs is quite similar as documented on the spectra of the bi-antennary glycopeptide SWPAVGNCSSALR (SEQ ID NO:28) of hemopexin (FIGS. 1A-1B). The spectra of both the fucosylated (FIG. 1A) and non-fucosylated (FIG. 1B) glycoforms contains Y-ions (m/z 1404.6 fucosylated and m/z 1331.6 non-fucosylated) that represent the loss of an arm (singly charged HexNAc-Hex m/z 366.1) and Y-ions (m/z 1323.6 and m/z 1250.5) from the loss of one arm with mannose (singly charged HexNAc-Hex-Hex m/z 528.2) visible in the low mass end. The m/z 366.1 oxonium ions are not seen because the spectra are acquired from 400 Da due to optimized ion transmission for the Y-ions.

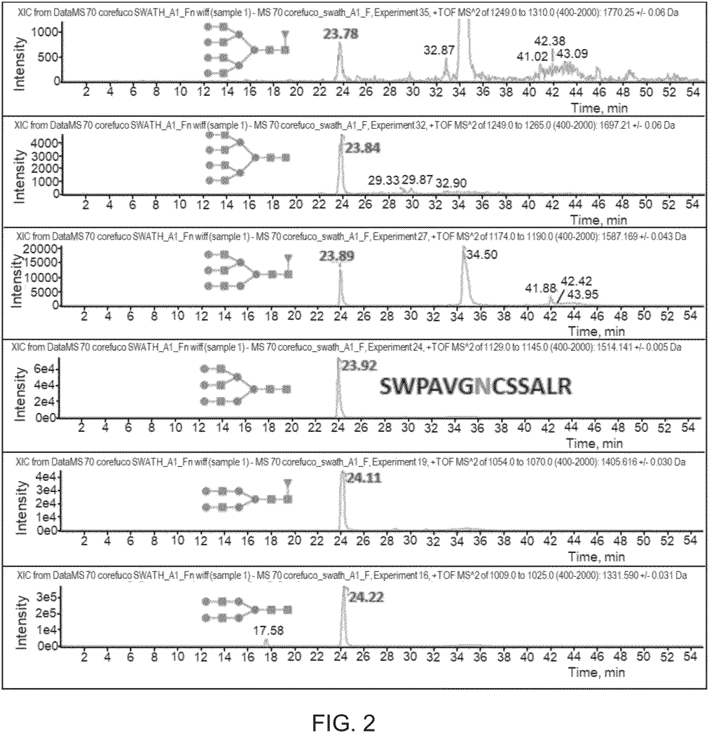

The low CE fragmentation allows efficient extraction of the product ions for quantification of the glycoforms as documented on the SWPAVGNCSSALR (SEQ ID NO: 28) glycopeptide of hemopexin (FIG. 2). The XIC chromatograms show the expected trend of slight decrease in the retention time (RT) of the glycoforms with the addition of each neutral carbohydrate unit as described previously.³⁸ The RT alignment further confirms the identity of the quantified analytes. The low CE settings also minimize the potential rearrangement of fucose which keeps the error in quantification of the fucosylated glycoform below 15%^{37,39} and maximizes the ion intensity sufficiently for reliable quantification of even the low-abundant tetra-antennary glycoforms.

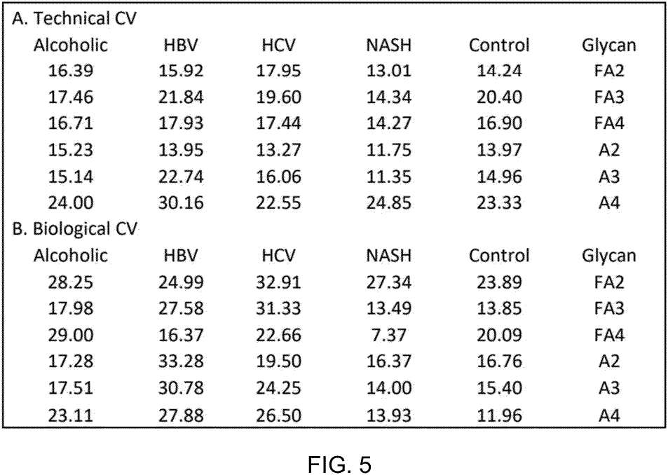

This is documented by the evaluation of the coefficients of variation (CV) across all the 90 glycopeptides quantified; the results are summarized as average CVs in the respective disease groups and glycoform classes (FIG. 5). Every sample was measured in duplicate and the duplicate measurements were compared to derive the technical CVs (FIG. 5, Technical CV). The average technical CVs of all fucosylated or non-fucosylated glycopeptides are 16.9% and 18.2%, respectively, and they are not statistically different (one-way ANOVA p=0.925). This analysis shows that the technical CVs do not differ between the disease groups but the CVs of tetraantennary nonfucosylated glycoforms are higher compared to the tri- or bi-antennary analytes. Interestingly technical CVs of fucosylated tetraantennary glycoforms does not follow this trend even if they are more abundant than nonfucosylated and influence of noise could be lower. As expected, the biological replicates (two different samples per group) show similar CVs with average CV 23.3% for the fucosylated glycopeptides compared to the average CV of 22.0% for the non-fucosylated glycopeptides (one-way ANOVA p=0.216). A significant difference was not observed in the biological CVs by disease group (one-way ANOVA p=0.091) or by the number of arms (one-way ANOVA p=0.445). Higher biological variability, observed especially in the disease groups, is expected and is minor compared to the fold-changes observed between the healthy controls and the cirrhotic disease groups (see below).

### Trends in the Abundance of Fucosylated and Non-Fucosylated Glycopeptides

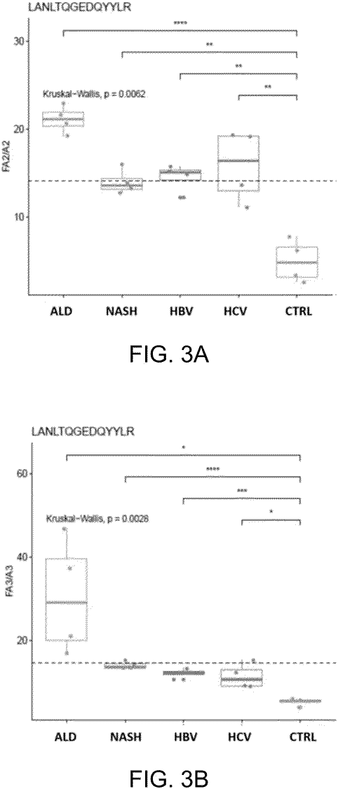

The core fucosylated glycopeptides show a general trend to increased intensities in the cirrhotic groups compared to the healthy controls (Table 2). When the peak areas of the same fucosylated glycoforms in the cirrhosis groups are compared to the healthy controls, 23 of 45 glycopeptides have higher intensity in cirrhosis; 6 of the glycopetides show significantly higher intensity in all four cirrhosis categories (p<0.05) compared to the healthy controls and all of them have intensity consistently more than 2-fold higher. Only 2 core fucosylated glycopeptides show consistently lower intensities in the four cirrhosis groups compared to the healthy controls. The highest difference in the peak areas of the fucosylated glycopeptide was observed for the tetra-antennary glycoform of the ELHHLQEQNVSNAFLDK (SEQ ID NO: 7) glycopeptide of ceruloplasmin which is on average 7.5 fold higher in the cirrhosis groups. One of the consistently and significantly changed core fucosylated glycopeptides is the biantennary and tri-antennary LANLTQGEDQYYLR (SEQ ID NO: 12) peptide of clusterin (FIGS. 3A-3B) which was previously described in liver disease³³ and by others (panel of analytes in patent US20090208926A1).

At the same time, a trend to lower intensities of the non-fucosylated glycopeptides in the cirrhosis groups was observed compared to the healthy controls (Table 2). We observe a consistently lower intensity of 14 glycopeptides in the four cirrhosis groups (p<0.05) and one of them has intensities decreased more than 2-fold. Only 3 non-fucosylated glycopeptides show significantly higher intensity in at least 2 of the cirrhosis groups. This trend reflects to some degree the reported trends in the serum concentration of the proteins in liver cirrhosis. Lower intensities were observed in the non-fucosylated glycopeptides of proteins (hemopexin, clusterin, ITIH4, fibrinogen) reported to decrease in cirrhosis while the nonfucosylated glycopeptide of alpha-2 macroglobulin has higher intensity in cirrhosis in line with its increased serum concentration.⁴⁰⁻⁴² However, additional factors affect the abundance of the non-fucosylated glycopeptides because some glycoforms of the same peptide show inconsistent trends. For example, the tri- and tetra-antennary glycoforms of some glycopeptides have higher intensity in cirrhosis while their bi-antennary glycoforms are lower. This trend may be potentially explained by increased branching in the cirrhosis groups. This is likely the case of the ELHHLQEQNVSNAFLDK (SEQ ID NO: 7) glycopeptide of ceruloplasmin where the tetra-antennary nonfucosylated glycoform has higher intensity in the cirrhosis groups while the bi-antennary glycoform is slightly lower in cirrhosis than in the healthy controls.

These trends suggest that the abundance of the core fucosylated glycoforms of some proteins goes up in cirrhosis in spite of the fact that the protein’s concentration may decrease. It is also a reason why the fucosylated glycoforms were normalized by their non-fucosylated counterparts and considered ratio as the two glycoforms as the final measure of interest.

### Structure Specific Changes of Core Fucosylation in Liver Cirrhosis

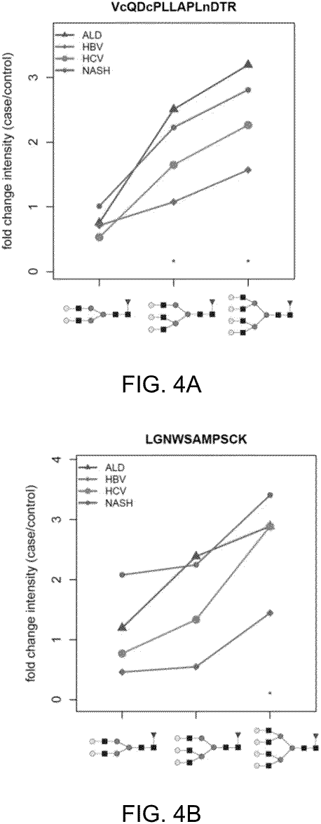

Multiple core fucosylated glycoproteins with significantly higher intensities of fucosylated glycopeptides are found in the serum of some category of the cirrhotic disease. Core fucosylated glycoforms (normalized intensity) of 12 glycopeptides of these proteins increase significantly (p<0.05) 3-fold or greater in at least two categories of liver cirrhosis compared to the healthy controls (Table 1). The normalized fucosylated intensities represented by the ratios of the same fucosylated/non-fucosylated glycoforms of a glycopeptide (i.e., bi-antennary fucosylated/bi-antennary non-fucosylated, etc.) were compared. Overall, the highest (5- to 9-fold) increase in the normalized fucosylated intensity for the bi-antennary glycoform of the QVFPGLNYCTSGAYSNASSTDSASYYPLTGD (SEQ ID NO: 27) glycopeptide of apolipoprotein B-100 was observed; the increase is significant in all the cirrhosis groups compared to the healthy control group. However, a decrease in the normalized fucosylated intensities for some glycopeptides were also observed. For example, the bi-antennary FGCEIENNR (SEQ ID NO: 30) glycopeptide of Zn-alpha2- glycoprotein has significantly lower normalized fucosylated intensities in the cirrhosis groups (Table 2) in line with previous reports of site- and structure-specific changes in the fucosylation of serum proteins in the context of liver cirrhosis.^{35,43} In addition, an interesting trend towards higher intensities of branched core fucosylated glycoforms for several peptides was observed. For example, the fucosylated intensities of the tri-antennary glycoform of the SWPAVGNCSSALR (SEQ ID NO: 28) peptide of hemopexin increase in cirrhosis consistently 2- to 5-fold while the intensities of the bi-antennary glycoforms increase 1-to 3-fold. Equally interesting are the trends of the bi-, tri- and tetra-antennary glycoforms of the VCQDCPLLAPLNDTR (SEQ ID NO: 29) peptide of Alpha-2-HS-glycoprotein and the LGNWSAMPSCK (SEQ ID NO: 26) glycopeptide of beta-2-glycoprotein (FIGS. 4A-4B). For all three glycopeptides, the increase in the intensity of the fucosylated analyte goes up with branching. The reason for this observation is unknown; it could be related to different rates of synthesis or to the post-synthetic selection of the fucosylated glycoforms. Nonetheless, the results show clearly that the presented DIA LC-MS/MS workflow enables quantification of the partially resolved fucosylated structures which would not be achieved with the commonly used truncation of the glycoforms with endoglycosidases.

TABLE 1

Normalized intensities of the core fucosylated glycoforms in healthy controls and in patients with cirrhosis of the liver of alcoholic (ALD), hepatitis B viral (HBV), hepatitis C viral (HCV), and non-alcoholic steatohepatitis (NASH) etiologies for glycopeptides with ratio of the glycoforms increased more than threefold in at least two categories of the cirrhotic liver disease compared to the healthy controls

| Peptide | SEQ ID NO: | Accession Number | Glycan ratio | Healthy | ALD | HBV | HCV | NASH | p-value | FDR |
|---|---|---|---|---|---|---|---|---|---|---|
| ELHHLQEQVSNAFLDK | 7 | P00450 | FA2/A2 | 0.738 | 3.52 | 2.456 | 2.526 | 2.479 | 0.026 | 0.034 |
| ELHHLQEQNVSNAFLDK | 7 | P00450 | FA3/A3 | 19.325 | 95.319 | 79.132 | 52.152 | 50.825 | 0.002 | 0.01 |
| ELHHLQEQNVSNAFLDK | 7 | P00450 | FA4/A4 | 33.133 | 152.706 | 190.84 | 106.261 | 101.359 | 0.003 | 0.01 |
| LANLTQGEDQYYLR | 12 | P01042 | FA2/A2 | 5.022 | 21.101 | 14.544 | 15.818 | 14.005 | 0.006 | 0.012 |
| LANLTQGEDQYYLR | 12 | P01042 | FA4/A 4 | 4.428 | 21.234 | 12.066 | 8.596 | 8.51 | 0.004 | 0.01 |
| LGNWSAMPSCK | 26 | P02751 | FA4/A4 | 26.469 | 118.536 | 259.891 | 40.991 | 37.053 | 0.022 | 0.03 |
| LDAPTNLQFVNETDSTVLVR | 13 | P02751 | FA2/A 2 | 9.33 | 29 | 38.24 | 28.22 | 35.39 | 0.031 | 0.039 |
| LGNWSAMPSCK | 26 | P02751 | FA3/A3 | 13.522 | 93.666 | 19.9 | 31.225 | 33.208 | 0.007 | 0.013 |
| LPTQNITFQTESSVAEQEAEFQSPK | 16 | Q14624 | FA4/A4 | 35.121 | 155.44 | 395.277 | 107.626 | 72.535 | 0.002 | 0.01 |
| QVFPGLNYCTSGAYSNASSTDSASYYPLTGD | 27 | P04114 | FA2/A2 | 0.749 | 6.845 | 3.681 | 5.007 | 4.264 | 0.015 | 0.023 |
| VDKDLQSLEDILHQVENK | 23 | P02671 | FA2/A2 | 21.869 | 66.857 | 44.836 | 81.947 | 67.083 | 0.007 | 0.013 |

“FAn/An”: ratio of the fucosylated glycopeptide to the nonfucosylated glycopeptide, “n” = number of antennas. “FDR” represents the false discovery rate.

TABLE 2

Summary of DIA results of selected fucosylated glycopeptides in liver disease

| ID | Uniprot ID | Protein Name | Gene | Peptide sequence | SEQ ID NO: |
|---|---|---|---|---|---|
| - | - | - | - | | |
| 1 | P02765 | Alpha-2-HS-glycoprotein | AHSG | AALAAFNAQNNGSNFQLEEISR | 1 |
| 2 | Q14624 | Inter-alpha-trypsin inhibitor heavy chain H4 | ITIH4 | AFITNFSMIIDGMTYPGIIK | 2 |
| 3 | P02790 | Hemopexin | HPX | ALPQPQNVTSLLGC(cam)TH | 3 |
| 4 | P027 90 | Hemopexin | HPX | ALPQPQNVTSLLGC(cam)TH | 3 |
| 5 | P027 90 | Hemopexin | HPX | ALPQPQNVTSLLGC(cam)TH | 3 |
| 6 | P02679 | Fibrinogen gamma chain | FGG | DLQSLEDILHQVENK | 4 |
| 7 | P02751 | Fibronectin | FN1 | DQC(cam)IVDDITYNVNDTFHK | 5 |
| 8 | P027 51 | Fibronectin | FN1 | DQC(cam)IVDDITYNVNDTFHK | 5 |
| 9 | P00450 | Ceruloplasmin | CP | EHEGAIYPDNTTDFQR | 6 |
| 10 | P00450 | Ceruloplasmin | CP | EHEGAIYPDNTTDFQR | 6 |
| 11 | P00450 | Ceruloplasmin | CP | EHEGAIYPDNTTDFQR | 6 |
| 12 | P00450 | Ceruloplasmin | CP | ELHHLQEQNVSNAFLDK | 7 |
| 13 | P00450 | Ceruloplasmin | CP | ELHHLQEQNVSNAFLDK | 7 |
| 14 | P00450 | Ceruloplasmin | CP | ELHHLQEQNVSNAFLDK | 7 |
| 15 | P25311 | Zn-alpha2-glycoprotein | AZGP1 | FGC(cam)EIENNR | 8 |
| 16 | P010 11 | alpha1-antichymotrypsin | SERPI NA3 | FNLTETSEAEIHQSFQHLLR | 9 |
| 17 | P198 27 | Inter-alpha-trypsin inhibitor heavy chain H1 | ITIH1 | IC(cam)DLLVANNHFAHFFAPQNLTNMNK | 10 |
| 18 | P08603 | Complement factor H | CFH | ISEENETTC(cam)YMGK | 11 |
| 19 | P10909 | Clusterin | CLU | LANLTQGEDQYYLR | 12 |
| 20 | P10909 | Clusterin | CLU | LANLTQGEDQYYLR | 12 |
| 21 | P10909 | Clusterin | CLU | LANLTQGEDQYYLR | 12 |
| 22 | P02751 | Fibronectin | FN1 | LDAPTNLQFVNETDSTVLVR | 13 |
| 23 | P01008 | Antithrombin-III | SERPINC1 | LGAC(cam)NDTLQQLMEVFK | 14 |
| 24 | P01008 | Antithrombin-III | SERPINC1 | LGAC(cam)NDTLQQLMEVFK | 14 |
| 25 | P02747 | beta-2-glycoprotein | APOH | LGNWSAMPSC(cam)K | 15 |
| 26 | P02747 | beta-2-glycoprotein | APOH | LGNWSAMPSC(cam)K | 15 |
| 27 | P02747 | beta-2-glycoprotein | APOH | LGNWSAMPSC(cam)K | 15 |
| 28 | Q146 24 | Inter-alpha-trypsin inhibitor heavy chain H4 | ITIH4 | LPTQNITFQTESSVAEQEAEFQSPK | 16 |
| 29 | Q14624 | Inter-alpha-trypsin inhibitor heavy chain H4 | ITIH4 | LPTQNITFQTESSVAEQEAEFQSPK | 16 |
| 30 | Q14624 | Inter-alpha-trypsin inhibitor heavy chain H4 | ITIH4 | LPTQNITFQTESSVAEQEAEFQSPK | 16 |
| 31 | H0YAC1 | Plasma kallikrein | KLKB1 | LQAPLNYTEFQKPIC(cam)LPSK | 17 |
| 32 | H0YAC1 | Plasma kallikrein | KLKB 1 | LQAPLNYTEFQKPIC(cam)LPSK | 17 |
| 33 | P05546 | heparin cofactor 2 precursor | SERPIND1 | NLSMPLLPADFHK | 18 |
| 34 | P041 14 | Apolipoprotein B-100 | APOB | QVFPGLNYC(cam)TSGAYSNASSTDSASYYPLTGDTR | 19 |
| 35 | P01008 | Antithrombin-III | SERPINC1 | SLTFNETYQDISELVYGAK | 20 |
| 36 | P02790 | Hemopexin | HPX | SWPAVGNC(cam)SSALR | 21 |
| 37 | P02790 | Hemopexin | HPX | SWPAVGNC(cam)SSALR | 21 |
| 38 | P02790 | Hemopexin | HPX | SWPAVGNC(cam)SSALR | 21 |
| 39 | P02765 | Alpha-2-HS-glycoprotein | AHSG | VC(cam)QDC(cam)PLLAPLNDTR | 22 |
| 40 | P02765 | Alpha-2-HS-glycoprotein | AHSG | VC(cam)QDC(cam)PLLAPLNDTR | 22 |
| 41 | P02765 | Alpha-2-HS-glycoprotein | AHSG | VC(cam)QDC(cam)PLLAPLNDTR | 22 |
| 42 | P02679 | Fibrinogengamma chain | FGG | VDKDLQSLEDILHQVENK | 23 |
| 43 | E9PGN7 | Plasma protease C1 inhibitor | SERPING1 | VGQLQLSHNLSLVILVPQNLK | 24 |
| 44 | P01023 | Alpha-2-macroglobulin | A2M | VSNQTLSLFFTVLQDVPVR | 25 |

The term “C(cam)” above refers to cysteine carbamidomethyl.

TABLE 2 continued

| ID | Glycan composition (aftertreatment with exoglycosidases) glycanwith/out core fucose | m/zprecursor nonfucosylated | m/zprecursor fucosylated | charge(z)precursor | Quantificationfragmentnon-gucosylated | Quantification fragment fucosylated | RT(min) |
|---|---|---|---|---|---|---|---|
| - | | | | | | | |
| 1 | HexNAc4Hex5Fuc/HexNAc4Hex5 | 1329.916 | 1378.602 | 3 | 1811.805 | 1884.834 | 34.49 |
| 2 | HexNAc4Hex5Fuc/HexNAc4Hex5 | 1285.58 | 1334.266 | 3 | 1745.3 | 1818.329 | 34.39 |
| 3 | HexNAc4Hex5Fuc/HexNAc4Hex5 | 1120.158 | 1168.844 | 3 | 1497.168 | 1570.197 | 32.22 |
| 4 | HexNAc5Hex6Fuc/HexNAc5Hex6 | 1241.869 | 1290.555 | 3 | 1679.734 | 1752.763 | 32.05 |
| 5 | HexNAc6Hex7Fuc/HexNAc6Hex7 | 1363.58 | 1412.266 | 3 | 1862.3 | 1935.329 | 31.95 |
| 6 | HexNAc4Hex5Fuc/HexNAc4Hex5 | 1135.168 | 1183.854 | 3 | 1519.682 | 1592.711 | 19.76 |
| 7 | HexNAc4Hex5Fuc/HexNAc4Hex5 | 1273.861 | 1322.547 | 3 | 1727.721 | 1800.75 | 32.68 |
| 8 | HexNAc5Hex6Fuc/HexNAc5Hex6 | 1395.572 | 1444.258 | 3 | 1910.288 | 1983.317 | 32.67 |
| 9 | HexNAc4Hex5Fuc/HexNAc4Hex5 | 879.611 | 916.125 | 4 | 1050.768 | 1099.454 | 19.5 |
| 10 | HexNAc5Hex6Fuc/HexNAc5Hex6 | 970.894 | 1007.409 | 4 | 1172.479 | 1221.165 | 19.31 |
| 11 | HexNAc6Hex7Fuc/HexNAc6Hex7 | 1062.177 | 1098.692 | 4 | 1294.19 | 1342.876 | 19.12 |
| 12 | HexNAc4Hex5Fuc/HexNAc4Hex5 | 911.902 | 948.416 | 4 | 1093.823 | 1142.508 | 18.45 |
| 13 | HexNAc5Hex6Fuc/HexNAc5Hex6 | 1003.185 | 1039.699 | 4 | 1215.533 | 1264.219 | 18.32 |
| 14 | HexNAc6Hex7Fuc/HexNAc6Hex7 | 1094.4678 | 1130.982 | 4 | 1337.244 | 1385.93 | 18.21 |
| 15 | HexNAc5Hex6Fuc/HexNAc5Hex6 | 1042.741 | 1091.427 | 3 | 1381.042 | 1454.071 | 28.13 |
| 16 | HexNAc5Hex6Fuc/HexNAc5Hex6 | 1097.733 | 1134.247 | 4 | 1341.6 | 1390.283 | 39.42 |
| 17 | HexNAc4Hex5Fuc/HexNAc4Hex5 | 911.207 | 940.418 | 5 | 1047.474 | 1083.988 | 30.21 |
| 18 | HexNAc4Hex5Fuc/HexNAc4Hex5 | 1062.082 | 1110.768 | 3 | 1410.054 | 1483.083 | 16.83 |
| 19 | HexNAc4Hex5Fuc/HexNAc4Hex5 | 1102.81 | 1151.496 | 3 | 1471.145 | 1544.174 | 26.37 |
| 20 | HexNAc5Hex6Fuc/HexNAc5Hex6 | 1224.52 | 1273.207 | 3 | 1654.711 | 1726.74 | 26.11 |
| 21 | HexNAc6Hex7Fuc/HexNAc6Hex7 | 1346.231 | 1394.917 | 3 | 1836.277 | 1909.306 | 25.96 |
| 22 | HexNAc4Hex5Fuc/HexNAc4Hex5 | 964.438 | 1000.953 | 4 | 1163.871 | 1212.557 | 34.4 |
| 23 | HexNAc4Hex5Fuc/HexNAc4Hex5 | 1163.835 | 1212.521 | 3 | 1562.683 | 1635.711 | 45.74 |
| 24 | HexNAc5Hex6Fuc/HexNAc5Hex6 | 1285.546 | 1334.232 | 3 | 1745.249 | 1818.278 | 45.29 |
| 25 | HexNAc4Hex5Fuc/HexNAc4Hex5 | 958.386 | 1007.072 | 3 | 1254.509 | 1327.538 | 22.59 |
| 26 | HexNAc5Hex6Fuc/HexNAc5Hex6 | 1080.097 | 1128.782 | 3 | 1437.075 | 1510.104 | 22.59 |
| 27 | HexNAc6Hex7Fuc/HexNAc6Hex7 | 1201.807 | 1250.493 | 3 | 1619.641 | 1692.67 | 22.35 |
| 28 | HexNAc4Hex5Fuc/HexNAc4Hex5 | 1108.74 | 1145.253 | 4 | 1356.272 | 1404.958 | 32.07 |
| 29 | HexNAc5Hex6Fuc/HexNAc5Hex6 | 1200.022 | 1236.536 | 4 | 1477.983 | 1526.669 | 31.87 |
| 30 | HexNAc6Hex7Fuc/HexNAc6Hex7 | 1291.305 | 1327.82 | 4 | 1599.694 | 1648.38 | 31.72 |
| 31 | HexNAc4Hex5Fuc/HexNAc4Hex5 | 968.197 | 1004.711 | 4 | 1168.882 | 1217.569 | 34.05 |
| 32 | HexNAc5Hex6Fuc/HexNAc5Hex6 | 1059.48 | 1095.994 | 4 | 1290.593 | 1339.279 | 33.85 |
| 33 | HexNAc4Hex5Fuc/HexNAc4Hex5 | 1035.791 | 1084.477 | 3 | 1370.617 | 1443.646 | 34.35 |
| 34 | HexNAc4Hex5Fuc/HexNAc4Hex5 | 1294.043 | 1330.558 | 4 | 1603.345 | 1652.031 | 35.04 |
| 35 | HexNAc4Hex5Fuc/HexNAc4Hex5 | 1267.552 | 1316.238 | 3 | 1718.258 | 1791.287 | 46.19 |
| 36 | HexNAc4Hex5Fuc/HexNAc4Hex5 | 1009.755 | 1058.441 | 3 | 1331.563 | 1404.591 | 24.22 |
| 37 | HexNAc5Hex6Fuc/HexNAc5Hex6 | 1131.466 | 1180.151 | 3 | 1514.129 | 1587.158 | 23.92 |
| 38 | HexNAc6Hex7Fuc/HexNAc6Hex7 | 1253.1763 | 1301.862 | 3 | 1697.695 | 1769.724 | 23.78 |
| 39 | HexNAc4Hex5Fuc/HexNAc4Hex5 | 1132.147 | 1180.833 | 3 | 1515.154 | 1588.18 | 29.71 |
| 40 | HexNAc5Hex6Fuc/HexNAc5Hex6 | 1253.86 | 1302.54 | 3 | 1697.72 | 1770.75 | 29.39 |
| 41 | HexNAc6Hex7Fuc/HexNAc6Hex7 | 1375.57 | 1424.25 | 3 | 1880.28 | 1953.31 | 29.38 |
| 42 | HexNAc4Hex5Fuc/HexNAc4Hex5 | 937.1752 | 973.69 | 4 | 1127.52 | 1176.206 | 27.08 |
| 43 | HexNAc4Hex5Fuc/HexNAc4Hex5 | 1312.653 | 1361.334 | 3 | 1785.911 | 1858.934 | 42.36 |
| 44 | HexNAc4Hex5Fuc/HexNAc4Hex5 | 1262.592 | 1311.278 | 3 | 1710.818 | 1783.848 | 55.74 |
| 45 | HexNAc6Hex7Fuc/HexNAc6Hex7 | 1384.303 | 1433 | 3 | 1893.385 | 1966.414 | 55.23 |

TABLE 2 continued

| ID | Intensity Healthy | Intensity ALD | Intensity HBV | Intensiy HCV | Intensity NASH | Intensity Healthy | Intensity ALD | Intensity HBV | Intensity HCV | Intensiy NASH |
|---|---|---|---|---|---|---|---|---|---|---|
| - | Fucosylated | | | | | Non-Fucosylated | | | | |
| 1 | 1124213 | 1237895 | 957536 | 1258819 | 1794829 | 9562146 | 5295234 | 5711101 | 7485535 | 6899279 |
| 2 | 222290.9 | 336252.8 | 224172.1 | 240186.8 | 348510.5 | 3370989 | 1667573 | 1751785 | 1637204 | 2014068 |
| 3 | 358863 | 502478.6 | 261153.6 | 275638.5 | 570737.6 | 33070569 | 15871837 | 18340065 | 18797898 | 26123871 |
| 4 | 24787.56 | 21756.85 | 13979.42 | 13263.82 | 25608.41 | 2930079 | 1225158 | 1403592 | 1805331 | 2207123 |
| 5 | 9160.63 | 10837.99 | 6689.204 | 10635.3 | 12344.65 | 153371.8 | 54937.93 | 60716.73 | 83413.25 | 111397.1 |
| 6 | 46406.07 | 134156.3 | 58168.16 | 66571.94 | 125458.2 | 1999005 | 948753.8 | 1426899 | 1791230 | 2148461 |
| 7 | 15905.46 | 15056.4 | 13280.03 | 16464.83 | 26385.13 | 687523.3 | 241115 | 608744.4 | 304650 | 374592.1 |
| 8 | 16594.72 | 16875.31 | 12020.9 | 17880.75 | 23073.29 | 114830.5 | 45174.85 | 96704.84 | 56284.59 | 57963.59 |
| 9 | 1099902 | 1400967 | 1181306 | 1234467 | 1856934 | 1421770 | 1003633 | 1187269 | 1287453 | 1426811 |
| 10 | 36378.47 | 51039.93 | 43231.07 | 41169.8 | 76978.61 | 2209229 | 1255342 | 1771959 | 2630817 | 2209169 |
| 11 | 40618.12 | 27621.16 | 46102.86 | 25268.22 | 29474.88 | 134099.5 | 77332.33 | 125214.5 | 346787.8 | 178496.7 |
| 12 | 65313.62 | 191405.9 | 131598.3 | 151691.7 | 193211.5 | 8725942 | 5268838 | 5245145 | 6041450 | 7921395 |
| 13 | 166818.1 | 500385 | 397290.4 | 430844.7 | 489665.5 | 853779.6 | 537256.8 | 510262.4 | 866483 | 964781.8 |
| 14 | 6002.83 | 49231.36 | 42164.2 | 48557.26 | 47069.91 | 21052.51 | 33059.23 | 21952.02 | 50322.08 | 47929 |
| 15 | 108708.8 | 40864.21 | 48401.32 | 53217.63 | 76527.39 | 244040.1 | 178855.3 | 187351.1 | 297882.5 | 254187.5 |
| 16 | 188343.2 | 909450.5 | 269866.7 | 194958.5 | 389337.7 | 4248751 | 4381366 | 3264321 | 3007787 | 3617108 |
| 17 | 111580.2 | 84752.74 | 101153.8 | 99968.81 | 95903.15 | 47748.94 | 143806.5 | 96545.73 | 202715.4 | 197320.2 |
| 18 | 108203 | 200884.1 | 286774.7 | 70528.54 | 213901 | 1656826 | 1183029 | 1896553 | 759706.4 | 1266790 |
| 19 | 140960.2 | 194473.3 | 170119.1 | 186142 | 208252 | 2815893 | 918271 | 1164576 | 1148867 | 1493843 |
| 20 | 129909.2 | 158968.8 | 109131.6 | 128023.4 | 174219.2 | 2429536 | 595394 | 917207.6 | 1141123 | 1255543 |
| 21 | 10960.7 | 12733.24 | 12773.08 | 12371.49 | 13707.82 | 253740.8 | 66203.77 | 107682.9 | 145593.8 | 162198.1 |
| 22 | 13116.09 | 24452.96 | 25186.49 | 21238.63 | 35355.58 | 143424 | 83763.29 | 66548.75 | 79467.98 | 100230.6 |
| 23 | 96815.12 | 94736.97 | 104882.5 | 98662.69 | 70565.69 | 6975276 | 4881451 | 7215600 | 6501520 | 5892678 |
| 24 | 14348.93 | 23864.36 | 20572.07 | 21920.28 | 16437.44 | 231196.1 | 101441.5 | 246128.3 | 159223.4 | 239507 |
| 25 | 84008.55 | 100469.1 | 38520.73 | 64417.8 | 174606.5 | 10160568 | 4637672 | 3611285 | 6666697 | 10322577 |
| 26 | 38985.37 | 93065.39 | 21342.94 | 51947.79 | 87519.46 | 287061.6 | 116504 | 91136.97 | 181915.8 | 269229.6 |
| 27 | 11509.57 | 33250.57 | 16624.77 | 33242.36 | 39242.1 | 47560.45 | 34279.91 | 27779.53 | 88789.67 | 110468.9 |
| 28 | 94491.91 | 102663.1 | 34267.43 | 27580.73 | 29262.42 | 1174058 | 628902.7 | 725141.4 | 723153.7 | 891879.3 |
| 29 | 77934.93 | 149639 | 97153.68 | 57406.42 | 87521.83 | 913128.3 | 608322.8 | 601748.5 | 897062.3 | 880887.4 |
| 30 | 21470.56 | 40034.63 | 65667.65 | 33666.81 | 23237.4 | 61367.46 | 27607.49 | 17084.65 | 32839.86 | 33033.72 |
| 31 | 178349.6 | 44088.9 | 41627.57 | 60372.72 | 137576.7 | 1784943 | 341085 | 608842.6 | 625578.6 | 824867.3 |
| 32 | 24745.71 | 22009.63 | 32077.07 | 29112.24 | 33030.78 | 410233.2 | 145192.6 | 197712.6 | 335656.6 | 3105742 |
| 33 | 369487.5 | 260973.1 | 91111.11 | 206417.6 | 403239.3 | 5214607 | 1394984 | 1182128 | 2326775 | 3077508 |
| 34 | 4741.323 | 25888.34 | 11434.47 | 22129.84 | 23838.07 | 633791.4 | 452501.3 | 310162.1 | 489378.9 | 559074.2 |
| 35 | 29534.89 | 69845.5 | 33932.53 | 26628.29 | 43869.65 | 3388881 | 1523264 | 2190558 | 2091635 | 2259822 |
| 36 | 4001415 | 2854133 | 2329995 | 2213730 | 4125403 | 18725139 | 6896897 | 8872987 | 8429226 | 12180218 |
| 37 | 581478.1 | 598990.2 | 358282.1 | 425041.4 | 743661.8 | 6862788 | 2616843 | 3264934 | 4192918 | 4893616 |
| 38 | 50194.54 | 74730.98 | 44858.33 | 61539.21 | 106539 | 688750.4 | 237586.4 | 295193.3 | 524571.5 | 554298.2 |
| 39 | 56974.14 | 42961.52 | 40571.29 | 30153.78 | 57640.33 | 949498.4 | 547592.2 | 593393.4 | 645891.7 | 826903 |
| 40 | 146951.5 | 368503.7 | 157980.5 | 242224.7 | 327213.1 | 4123458 | 2328977 | 2372881 | 4019686 | 3397923 |
| 41 | 16232.11 | 51846.21 | 25500.91 | 36754.27 | 45598.26 | 204441.7 | 118042.1 | 134992.8 | 231901.4 | 227352.5 |
| 42 | 79862.32 | 85159.43 | 75367.18 | 163109.5 | 150468.3 | 370292.7 | 125191.2 | 168919.9 | 200881.4 | 229299.5 |
| 43 | 36021.83 | 33956.68 | 40458.39 | 44536.04 | 39867.16 | 663713.4 | 307441.2 | 325468 | 468288.3 | 361190.3 |
| 44 | 42311.65 | 76258.23 | 119544.2 | 152381.7 | 112594.5 | 133431.5 | 273346.9 | 417969.3 | 811238.2 | 594753.7 |
| 45 | 11499.93 | 18969.14 | 25957.01 | 42820.29 | 36898.41 | 16054.5 | 11685.41 | 20261.73 | 60850.07 | 35414.81 |

### Discussion

Knowledge of the extent of core fucosylation of human proteins substantially expanded in recent years.²⁸⁻³³ The reported studies use a variety of enrichment strategies and mass spectrometric workflows to improve coverage of the site-specific core fucosylated proteins but quantification of the core fucosylated analytes remains challenging.²⁸⁻³³ This is due to some extent to the complex nature of quantification of the glycoforms at specific sites of attachment to proteins⁴⁴ but also due to the fact that synthetic isotopically labeled standards are not available for the quantification of glycopeptides. The studies therefore focus on relative quantification using iTRAQ or TMT labeling³⁰⁻³² which is often combined with truncation of the N-glycans with endoglycosidases^{29,31} to improve identification and relative quantification of the truncated (peptide with GlcNAc with/out fucose) analytes. This is, however, by no means straightforward as the efficiency of cleavage of the iTRAQ or TMT tags is affected by the presence of complex glycans and because the rate of cleavage of some types of complex glycans (fucosylated, branched) by endoglycosidases differs substantially (some are not appreciably cleaved). As a consequence, several thousand sites modified to some extent by core fucosylated N-glycans are known, but their relative intensities are known to a limited extent.²⁸⁻³³

In this study, a workflow was introduced that eliminates the endoglycosidase digest and extends the glycopeptide SWATH (GP-SWATH) analyses, that was introduced recently³⁵, to relative quantification of the core fucosylated glycopeptides. Exoglycosidase digests were combined with DIA LC-MS/MS using low CE fragmentation, which enables quantification of the core fucosylated glycopeptides with partially resolved N-glycan structures, namely branching into bi-, tri-, and tetra-antennary glycoforms. This is important because the knowledge of the core fucose distribution on glycoproteins by their branching is limited.

Exoglycosidases (α2-3,6,8,9 neuraminidase, 1-3,4 fucosidase and 1-2 fucosidase) were used because the focus was on the core fucose. The outer-arm modifications dilute the MS signal into multiple low abundant analytes which may have overlapping isotope clusters⁴⁵; removal of the outer-arm fucose also simplifies data interpretation. Low CE fragmentation is an important component of the workflow because it minimizes interferences of peptides (which remain to a large extent unfragmented), eliminates excessive fragmentation of the glycans (FIGS. 1A-1B) and potential rearrangement of fucose as described recently.^{39,46} This set of optimizations makes the glycopeptide Y-ions sufficiently selective for reliable quantification of the branched (bi- to tetra-antennary) core fucosylated glycopeptides by the GP-SWATH DIA LC-MS/MS. These results document relative quantification of 45 glycopeptide pairs (with/out fucose) with average technical CV around 17% (FIG. 5) and demonstrate meaningful biological assessment of differences in the core fucosylation of serum glycoproteins in liver cirrhosis of various etiologies.

Higher core fucosylation is typically observed in liver cirrhosis compared to the healthy controls; a typical increase is up to 2-fold but up to 8-fold increased intensity of the fucosylated glycoform was observed in the case of the tetra-antennary ELHHLQEQNVSNAFLDK (SEQ ID NO: 7) glycopeptide of ceruloplasmin, depending on the etiology of the cirrhosis. The increase in core fucosylation is more pronounced in the groups of cirrhosis of ALD (average: 1.79) and NASH (average 1.88) etiologies (15 and 21 of the 45 glycopeptides with significantly higher core fucosylation compared to the healthy controls, respectively) as opposed to HBV and HCV etiology (8 and 12 glycopeptides). At the same time, a trend towards lower intensity of the nonfucosylated glycoforms in cirrhosis was observed which is likely related, to some degree, to the decrease in concentration of some of the proteins in the cirrhotic patients. It is well known that synthesis of proteins in the liver is affected by the progressing fibrotic remodeling. The intensity of the fucosylated glycoforms was therefore normalized and the ratio of fucosylated to the nonfucosylated forms was used as the quantified readout (Table 1).

Some bi-antennary glycoppetides decrease in cirrhosis compared to healthy controls but increase in cirrhosis in the tri- or tetra-antennary forms of the same peptide. This cannot be explained by lower protein concentration but could perhaps be attributed to increased branching; further studies will be needed to define whether this originates at synthesis or post-synthetic selection. The VC(cam)QDC(cam)PLLAPLNDTR (SEQ ID NO: 22) glycopeptide of Alpha-2-HS-glycoprotein is a good example with higher contribution of tetra-antennary glycoforms in the cirrhosis groups while the bi-antennary and triantennary glycoforms remain the same. Relative contribution of the bi-, tri and tetra-antennary structures in the healthy group for this peptide is 18%, 78% and 4%, respectively and the ratio of the glycoforms in the cirrhotic groups compared to the healthy group range from 0.71 to 1.04 for the bi-antennary glycopeptides, 0.98 to 1.05 for tri-antennary, and 1.20 to 1.40 for the tetra-antennary glycopeptides. This is true for several glycopeptides (for example LGNWSAMPSCK (SEQ ID NO: 26)) and supports the usefulness of the analytical approach where the increase of core fucosylation and increase of glycan branching are used simultaneously. The greater than 4-fold increases of the normalized intensities of some of the glycopeptides in cirrhosis suggests that the core fucosylated glycopeptides might be useful in serologic monitoring of liver fibrosis.

The workflow has an average technical CV of 17.5% and shows consistent trends which suggests good performance.

In summary, an exoglycosidase-assisted GP-SWATH analysis of core fucosylated glycopeptides with resolved branching of the N-glycans was introduced. The DIA LCMS/MS workflow identifies meaningful differences in the relative quantities of site-specific core fucosylated glycopeptides in a preliminary study of liver cirrhosis. The data suggest that the degree of core fucosylation and its disease-associated alterations differ by branching of the N-glycans as documented on the case of ceruloplasmin or hemopexin. The workflow will enable quantitative studies of core fucosylation in biological model systems and in liquid biopsies such as serum, urine, or cerebrospinal fluid (CSF).

### Cited References

1. Rudd, P. M., Elliott, T., Cresswell, P., Wilson, I. A. & Dwek, R. A. Glycosylation and the immune system. Science 291(5512), 2370-2376 (2001).

2. Helenius, A. & Aebi, M. Intracellular functions of N-linked glycans. Science 291(5512), 2364-2369 (2001).

3. Cummings, R. D. & Pierce, J. M. The challenge and promise of glycomics. Chem. Biol. 21(1), 1-15 (2014).

4. Hudak, J. E. & Bertozzi, C. R. Glycotherapy: new advances inspire a reemergence of glycans in medicine. Chem. Biol. 21(1), 16-37 (2014).

5. Varki, A., & Gagneux, P. Biological Functions of Glycans. 2015, 77-88.

6. Schneider, M., Al-Shareffi, E. & Haltiwanger, R. S. Biological functions of fucose in mammals. Glycobiology 27(7), 601-618 (2017).

7. Reily, C., Stewart, T. J., Renfrow, M. B. & Novak, J. Glycosylation in health and disease. Nat. Rev. Nephrol. 15(6), 346-366 (2019).

8. Li, J., Hsu, H. C., Mountz, J. D. & Allen, J. G. Unmasking fucosylation: From cell adhesion to immune system regulation and diseases. Cell Chem. Biol. 25(5), 499-512 (2018).

9. Miyoshi, E. et al. The alpha 1-6-fucosyltransferase gene and its biological significance. Biochim. Biophys. Acta 1473(1), 9-20 (1999).

10. Breton, C., Oriol, R. & Imberty, A. Conserved structural features in eukaryotic and prokaryotic fucosyltransferases. Glycobiology 8(1), 87-94 (1998).

11. Balog, C. I. et al. N-glycosylation of colorectal cancer tissues: a liquid chromatography and mass spectrometry-based investigation. Mol. Cell Proteomics. 11(9), 571-585 (2012).

12. Chandler, K. & Goldman, R. Glycoprotein disease markers and single protein-omics. Mol. Cell Proteomics. 12(4), 836-845 (2013).

13. Tjondro, H. C., Loke, I., Chatterjee, S. & Thaysen-Andersen, M. Human protein paucimannosylation: Cues from the eukaryotic kingdoms. Biol. Rev. Camb. Philos. Soc. 94(6), 2068-2100 (2019).

14. Wang, X., Gu, J., Miyoshi, E., Honke, K. & Taniguchi, N. Phenotype changes of Fut8 knockout mouse: Core fucosylation is crucial for the function of growth factor receptor(s). Methods Enzymol. 417, 11-22 (2006).

15. Ng, B. G. et al. Biallelic mutations in FUT8 cause a congenital disorder of glycosylation with defective fucosylation. Am. J. Hum. Genet. 102(1), 188-195 (2018).

16. Ferrara, C., Stuart, F., Sondermann, P., Brunker, P., & Umana, P. The carbohydrate at FcgammaRIIIa Asn-162. An element required for high affinity binding to non-fucosylated IgG glycoforms. J. Biol. Chem. 281 (8), 5032-5036 (2006).

17. Majewska, N. I., Tejada, M. L., Betenbaugh, M. J., & Agarwal, N. N-glycosylation of IgG and IgG-like recombinant therapeutic proteins: Why is it important and how can we control it? Annu. Rev. Chem. Biomol. Eng (2020).

18. Dekkers, G., Treffers, L., Plomp, R., Bentlage, A. E. H., de, B. M., Koeleman, C. A. M., Lissenberg-Thunnissen, S. N., Visser, R., Brouwer, M., Mok, J. Y., Matlung, H., van den Berg, T. K., van Esch, W. J. E., Kuijpers, T. W., Wouters, D., Rispens, T., Wuhrer, M., & Vidarsson, G. Decoding the human immunoglobulin G-glycan repertoire reveals a spectrum of Fc-receptor- and complement-mediated-effector activities. Front. Immunol. 2017, 8, 877.

19. Okazaki, A. et al. Fucose depletion from human IgGl oligosaccharide enhances binding enthalpy and association rate between IgGl and FcgammaRIIIa. J. Mol. Biol. 336(5), 1239-1249 (2004).

20. Liang, W. et al. Core fucosylation of the T cell receptor is required for T cell activation. Front Immunol. 9, 78 (2018).

21. Okada, M. et al. Blockage of core fucosylation reduces cell-surface expression of PD-1 and promotes anti-tumor immune responses of T cells. Cell Rep. 20(5), 1017-1028 (2017).

22. Agrawal, P. et al. A systems biology approach identifies FUT8 as a driver of melanoma metastasis. Cancer Cell 31 (6), 804-819 (2017).

23. Wang, Y. et al. Loss of alphal,6-fucosyltransferase inhibits chemical-induced hepatocellular carcinoma and tumorigenesis by down-regulating several cell signaling pathways. FASFB J. 29(8), 3217-3227 (2015).

24. Liu, Y. C., Yen, H. Y., Chen, C. Y., Chen, C. H., Cheng, P. F., Juan, Y. H., Chen, C. H., Khoo, K. H., Yu, C. J., Yang, P. C., Hsu, T. L., & Wong, C. H. Sialylation and fucosylation of epidermal growth factor receptor suppress its dimerization and activation in lung cancer cells. Proc. Natl. Acad. Sci. USA 108 (28), 11332-11337 (2011).

25. Honma, R. et al. Expression of fucosyltransferase 8 is associated with an unfavorable clinical outcome in non-small cell lung cancers. Oncology 88(5), 298-308 (2015).

26. Fujii, H. et al. Core fucosylation on T cells, required for activation of T-cell receptor signaling and induction of colitis in mice, is increased in patients with inflammatory bowel disease. Gastroenterology 150(7), 1620-1632 (2016).

27. Wang, T. T. et al. IgG antibodies to dengue enhanced for FcgammaRIIIA binding determine disease severity. Science 355(6323), 395-398 (2017).

28. Jia, W. et al. A strategy for precise and large scale identification of core fucosylated glycoproteins. Mol. Cell Proteomics. 8(5), 913-923 (2009).

29. Cao, Q. et al. Strategy integrating stepped fragmentation and glycan diagnostic ion-based spectrum refinement for the identification of core fucosylated glycoproteome using mass spectrometry. Anal. Chem. 86(14), 6804-6811 (2014).

30. Yin, H. et al. Mass-selected site-specific core-fucosylation of serum proteins in hepatocellular carcinoma. J. Proteome. Res. 14(11), 4876-4884 (2015).

31. Ma, C. et al. A precise approach in large scale core-fucosylated glycoprotein identification with low- and high-normalized collision energy. J. Proteomics. 114, 61-70 (2015).

32. Zhou, J. et al. Site-specific fucosylation analysis identifying glycoproteins associated with aggressive prostate cancer cell lines using tandem affinity enrichments of intact glycopeptides followed by mass spectrometry. Anal. Chem. 89(14), 7623-7630 (2017).

33. Ma, J., Sanda, M., Wei, R., Zhang, L. & Goldman, R. Quantitative analysis of core fucosylation of serum proteins in liver diseases by LC-MS-MRM. J. Proteomics. 189, 67-74 (2018).

34. Yuan, W., Benicky, J., Wei, R., Goldman, R. & Sanda, M. Quantitative analysis of sex-hormone-binding globulin glycosylation in liver diseases by liquid chromatography-mass spectrometry parallel reaction monitoring. J. Proteome. Res. 17(8), 2755-2766 (2018).

35. Sanda, M., Zhang, L., Edwards, N. J. & Goldman, R. Site-specific analysis of changes in the glycosylation of proteins in liver cirrhosis using data-independent workflow with soft fragmentation. Anal. Bioanal. Chem. 409(2), 619-627 (2016).

36. Kozlik, P., Goldman, R. & Sanda, M. Hydrophilic interaction liquid chromatography in the separation of glycopeptides and their isomers. Anal. Bioanal. Chem. 410(20), 5001-5008 (2018).

37. Sanda, M. & Goldman, R. Data independent analysis of IgG glycoforms in samples of unfractionated human plasma. Anal. Chem. 88(20), 10118-10125 (2016).

38. Kozlik, P., Goldman, R. & Sanda, M. Study of structure-dependent chromatographic behavior of glycopeptides using reversed phase nanoLC. Electrophoresis 38(17), 2193-2199 (2017).

39. Acs, A., Ozohanics, O., Vekey, K., Drahos, L. & Turiak, L. Distinguishing core and antenna fucosylated glycopeptides based on low-energy tandem mass spectra. Anal. Chem. 90(21), 12776-12782 (2018).

40. Grieco, A. et al. Plasma levels of fibronectin in patients with chronic viral and alcoholic liver disease. Hepatogastroenterology 45(23), 1731-1736 (1998).

41. Niu, L., Geyer, P. E., Wewer Albrechtsen, N. J., Gluud, L. L., Santos, A., Doll, S., Treit, P. V., Holst, J. J., Knop, F. K., Vilsboll, T., Junker, A., Sachs, S., Stemmer, K., Muller, T. D., Tschop, M. H., Hofmann, S. M., & Mann, M. Plasma proteome profiling discovers novel proteins associated with non-alcoholic fatty liver disease. Mol. Syst. Biol. 15 (3), e8793 (2019).

42. Gangadharan, B., Antrobus, R., Dwek, R. A. & Zitzmann, N. Novel serum biomarker candidates for liver fibrosis in hepatitis C patients. Physiol. Rev 53(10), 1792-1799 (2007).

43. Benicky, J., Sanda, M., Pompach, P., Wu, J. & Goldman, R. Quantification of fucosylated hemopexin and complement factor h in plasma of patients with liver disease. Anal. Chem. 86(21), 10716-10723 (2014).

44. Goldman, R. & Sanda, M. Targeted methods for quantitative analysis of protein glycosylation. Proteomics. Clin. Appl. 9(1-2), 17-32 (2015).

45. Sanda, M. et al. Quantitative liquid chromatography-mass spectrometry-multiple reaction monitoring (LC-MS-MRM) analysis of site-specific glycoforms of haptoglobin in liver disease. Mol. Cell Proteomics. 12(5), 1294-1305 (2013).

46. Brockhausen, I., Möller, G., Merz, G., Adermann, K. & Paulsen, H. Control of mucin synthesis: The peptide portion of synthetic O-glycopeptide substrates influences the activity of O-glycan Core 1 UDPgalactose:N-acetyl-d-galactosaminyl-R β3-galactosaminyltransferase. Biochemistry 29, 10206-10212 (1990).

1. A method of detecting N-glycopeptides in a sample, the method comprising:
a) contacting the sample with one or more exoglycosidases, thereby producing an exoglycosidase-treated sample; and
b) detecting the N-glycopeptides in the exoglycosidase-treated sample by mass spectrometry.

2. The method of claim 1, wherein the exoglycosidase is a neuraminidase or a fucosidase, optionally wherein the neuraminidase is α2-3,6,8,9 neuraminidase, and/or wherein the fucosidase is α1-2 fucosidase or α1-3,4 fucosidase.

3-4. (canceled)

5. The method of claim 1, wherein the sample is contacted with at least one neuraminidase followed by at least one fucosidase, optionally wherein the sample is contacted with α2-3,6,8,9 neuraminidase followed by α1-2 fucosidase or α1-3,4 fucosidase, wherein the sample is contacted with α2-3,6,8,9 neuraminidase followed by α1-2 fucosidase and α1-3,4 fucosidase, wherein the sample is contacted with α2-3,6,8,9 neuraminidase at about 37° C. for about 8 to about 24 hours followed by α1-2 fucosidase and α1-3,4 fucosidase at about 37° C. for about 8 to about 24 hours, or wherein the sample is not contacted with an endoglycosidase.

6-9. (canceled)

10. The method of claim 1, wherein the sample is contacted with trypsin prior to contacting with the one or more exoglycosidases, or wherein the sample is contacted with an affinity reagent prior to contacting with the one or more exoglycosidases to remove a substantial portion of abundant proteins, optionally wherein the abundant proteins comprise one or more of albumin, IgG, antitrypsin, IgA, transferrin, haptoglobin, fibrinogen, alpha2-macroglobulin, alphal-acid glycoprotein, IgM, apolipoprotein AI, apolipoprotein AII, complement C3, and transthyretin, and/or wherein at least about 90% of the abundant proteins are removed.

11-13. (canceled)

14. The method of claim 1, wherein the mass spectrometry comprises liquid chromatography - tandem mass spectrometry (LC-MS-MS) or data-independent acquisition (DIA) LC-MS-MS, optionally wherein
the mass spectrometry is performed with low collision energy (CE) fragmentation, wherein the low CE fragmentation comprises less than about 50%, less than about 40%, less than about 30%, or less than about 10% normalized collision energy (NCE), wherein the mass spectrometry is performed with an MS1 scan of a resolution of about 30,000 to about 120,000 at about 400 to about 2000 m/z, and/or wherein the mass spectrometry is performed with an MS2 scan of a resolution of about 7,500 to about 30,000 at about 100 to about 2000 m/z; and/or
wherein the mass spectrometry detects core fucosylation of the N-glycopeptides, or wherein the mass spectrometry detects bi-, tri-, and tetra-antennary forms of a specific N-glycopeptide.

15-20. (canceled)

21. The method of claim 1, wherein the sample comprises serum, urine, or cerebrospinal fluid (CSF).

22. The method of claim 1, wherein the sample is isolated from a subject, optionally wherein the subject is suffering from a liver disease or suspected of having a liver disease, wherein the liver disease is liver cirrhosis, and/or wherein the liver cirrhosis is of an alcoholic (ALD), a hepatitis B viral (HBV), a hepatitis C viral (HCV), and/or a non-alcoholic steatohepatitis (NASH) etiology.

23-25. (canceled)

26. A method of detecting the presence or progression of a liver disease in a subject, the method comprising:
a) contacting a sample isolated from the subject with one or more exoglycosidases, thereby producing an exoglycosidase-treated sample; and
b) detecting in the exoglycosidase-treated sample by mass spectrometry one or both of:
i-a) core fucosylation of N-glycopeptides, and
ii-a) bi-, tri-, and tetra-antennary forms of a specific N-glycopeptide,
wherein:
i-b) an increase of core fucosylation relative to a control sample indicates the presence or progression of the liver disease in the subject, and
ii-b1) a decrease of the bi-antennary form of the specific N-glycopeptide relative to
a control sample indicates the presence or progression of liver disease in the subject, or
ii-b2) an increase of the tri- and/or tetra-antennary form of the specific N-glycopeptide relative to a control sample indicates the presence or progression of liver disease in the subject.

27. The method of claim 26, wherein an increase in the ratio of fucosylated bi-, tri-, or tetra-antennary forms of a specific N-glycopeptide to non-fucosylated bi-, tri-, or tetra-antennary forms of a specific N-glycopeptide relative to a control sample indicates the presence or progression of liver disease in the subject, optionally wherein the increase of core fucosylation relative to a control sample is at least about 1.5-fold.

28. (canceled)

29. The method of claim 26 , wherein the exoglycosidase is a neuraminidase or a fucosidase, optionally wherein the neuraminidase is α2-3,6,8,9 neuraminidase, and/or wherein the fucosidase is α1-2 fucosidase or α1-3,4 fucosidase.

30-31. (canceled)

32. The method of claim 26, wherein the sample is contacted with at least one neuraminidase followed by at least one fucosidase, optionally wherein the sample is contacted with α2-3,6,8,9 neuraminidase followed by α1-2 fucosidase or α1-3,4 fucosidase, wherein the sample is contacted with α2-3,6,8,9 neuraminidase followed by α1-2 fucosidase and α1-3,4 fucosidase, wherein the sample is contacted with α2-3,6,8,9 neuraminidase at about 37° C. for about 8 to about 24 hours followed by α1-2 fucosidase and α1-3,4 fucosidase at about 37° C. for about 8 to about 24 hours, or wherein the sample is not contacted with an endoglycosidase.

33-36. (canceled)

37. The method of claim 26, wherein the sample is contacted with trypsin prior to contacting with the one or more exoglycosidases, or wherein the sample is contacted with an affinity reagent prior to contacting with the one or more exoglycosidases to remove a substantial portion of abundant proteins, optionally wherein the abundant proteins comprise one or more of albumin, IgG, antitrypsin, IgA, transferrin, haptoglobin, fibrinogen, alpha2-macroglobulin, alphal-acid glycoprotein, IgM, apolipoprotein AI, apolipoprotein AII, complement C3, and transthyretin, and/or wherein at least about 90% of the abundant proteins are removed.

38-40. (canceled)

41. The method of claim 26, wherein the mass spectrometry comprises liquid chromatography - tandem mass spectrometry (LC-MS-MS), or data-independent acquisition (DIA) LC-MS-MS, optionally wherein the mass spectrometry is performed with low collision energy (CE) fragmentation, optionally wherein the low CE fragmentation comprises less than about 50%, less than about 40%, less than about 30%, or less than about 10% normalized collision energy (NCE), wherein the mass spectrometry is performed with an MS1 scan of a resolution of about 30,000 to about 120,000 at about 400 to about 2000 m/z, and/or wherein the mass spectrometry is performed with an MS2 scan of a resolution of about 7,500 to about 30,000 at about 100 to about 2000 m/z.

42-45. (canceled)

46. The method of claim 26, wherein the sample comprises serum.

47. The method of claim 26, wherein the liver disease is liver cirrhosis, optionally wherein the liver cirrhosis is of an alcoholic (ALD), a hepatitis B viral (HBV), a hepatitis C viral (HCV), and/or a non-alcoholic steatohepatitis (NASH) etiology.

48. (canceled)

49. The method of claim 26, wherein the N-glycopeptide comprises one or more of:
AALAAFNAQNNGSNFOLEEISR (SEQ ID NO: 1);
AFITNFSMIIDGMTYPGIIK (SEQ ID NO: 2);
ALPOPQNVTSLLGC(cam)TH (SEQ ID NO: 3);
DLQSLEDILHQVENK (SEQ ID NO: 4);
DQC(cam)IVDDITYNVNDTFHK (SEQ ID NO: 5);
EHEGAIYPDNTTDFOR (SEQ ID NO: 6);
ELHHLOEONVSNAFLDK (SEQ ID NO: 7);
FGC(cam)EIENNR (SEQ ID NO: 8);
FNLTETSEAEIHOSFQHLLR (SEQ ID NO: 9);
IC(cam)DLLVANNHFAHFFAPQNLTNMNK (SEQ ID NO: 10);
ISEENETTC(cam)YMGK (SEQ ID NO: 11);
LANLTQGEDQYYLR (SEQ ID NO: 12);
LDAPTNLQFVNETDSTVLVR (SEQ ID NO: 13);
LGAC(cam)NDTLQQLMEVFK (SEQ ID NO: 14);
LGNWSAMPSC(cam)K (SEQ ID NO: 15);
LPTQNITFQTESSVAEQEAEFQSPK (SEQ ID NO: 16);
LQAPLNYTEFQKPIC(cam)LPSK (SEQ ID NO: 17);
NLSMPLLPADFHK (SEQ ID NO: 18);
QVFPGLNYC(cam)TSGAYSNASSTDSASYYPLTGDTR (SEQ ID NO: 19);
SLTFNETYQDISELVYGAK (SEQ ID NO: 20);
SWPAVGNC(cam)SSALR (SEQ ID NO: 21);
VC(cam)QDC(cam)PLLAPLNDTR (SEQ ID NO: 22);
VDKDLQSLEDILHQVENK (SEQ ID NO: 23);
VGQLQLSHNLSLVILVPQNLK (SEQ ID NO: 24);
VSNQTLSLFFTVLQDVPVR (SEQ ID NO: 25);
LGNWSAMPSCK (SEQ ID NO: 26); and
QVFPGLNYCTSGAYSNASSTDSASYYPITGD (SEQ ID NO: 27).

50. (canceled)

51. A method of treating a liver disease in a subject, the method comprising:
a) contacting a sample isolated from the subject with one or more exoglycosidases, thereby producing an exoglycosidase-treated sample;
b) detecting in the exoglycosidase-treated sample by mass spectrometry one or both of:
i-a) core fucosylation of N-glycopeptides, and
ii-a) bi-, tri-, and tetra-antennary forms of a specific N-glycopeptide; and
c) administering a therapy to the subject to treat the liver disease,
wherein the therapy is administered if:
i-b) an increase of core fucosylation relative to a control sample is detected in the subj ect,
ii-b1) a decrease of the bi-antennary form of the specific N-glycopeptide relative to a control sample is detected in the subject, and/or
ii-b2) an increase of the tri- and/or tetra-antennary form of the specific N-glycopeptide relative to a control sample is detected in the subject.

52. The method of claim 51, wherein the therapy is useful to treat the liver disease, optionally wherein the liver disease is liver cirrhosis, and/or the liver cirrhosis is of an alcoholic (ALD), a hepatitis B viral (HBV), a hepatitis C viral (HCV), and/or a non-alcoholic steatohepatitis (NASH) etiology.

53-54. (canceled)

55. The method of claim 51, wherein the therapy comprises a liver cirrhosis therapy, optionally wherein the liver cirrhosis therapy comprises one or more of naltrexone, acamprosate, vitamin E, pioglitazone, an antiviral drug, an interferon, and ursodiol, wherein the antiviral drug is selected from the group consisting of entecavir, tenofovir, lamivudine, adefovir, and telbivudine, and/or wherein the interferon comprises interferon alpha 2b or pegylated interferon.

56-58. (canceled)

59. The method of claim 51, wherein the N-glycopeptide comprises one or more of:
AALAAFNAQNNGSNFQLEEISR (SEQ ID NO: 1);
AFITNFSMIIDGMTYPGIIK (SEQ ID NO: 2);
ALPOPQNVTSLLGC(cam)TH (SEQ ID NO: 3);
DLQSLEDILHQVENK (SEQ ID NO: 4);
DQC(cam)IVDDITYNVNDTFHK (SEQ ID NO: 5);
EHEGAIYPDNTTDFOR (SEQ ID NO: 6);
ELHHLOEONVSNAFLDK (SEQ ID NO: 7);
FGC(cam)EIENNR (SEQID NO: 8);
FNLTETSEAEIHOSFQHLLR (SEQ ID NO: 9);
IC(cam)DLLVANNHFAHFFAPQNLTNMNK (SEQ ID NO: 10);
ISEENETTC(cam)YMGK (SEQ ID NO: 11);
LANLTQGEDQYYLR (SEQ ID NO: 12);
LDAPTNLQFVNETDSTVLVR (SEQ ID NO: 13);
LGAC(cam)NDTLQQLMEVFK (SEQ ID NO: 14);
LGNWSAMPSC(cam)K (SEQ ID NO: 15);
LPTQNITFQTESSVAEQEAEFQSPK (SEQ ID NO: 16);
LQAPLNYTEFQKPIC(cam)LPSK (SEQ ID NO: 17);
NLSMPLLPADFHK (SEQ ID NO: 18);
QVFPGLNYC(cam)TSGAYSNASSTDSASYYPLTGDTR (SEQ ID NO: 19);
SLTFNETYQDISELVYGAK (SEQ ID NO: 20);
SWPAVGNC(cam)SSALR (SEQ ID NO: 21);
VC(cam)QDC(cam)PLLAPLNDTR (SEQ ID NO: 22);
VDKDLQSLEDILHQVENK (SEQ ID NO: 23);
VGQLQLSHNLSLVILVPQNLK (SEQ ID NO: 24);
VSNQTLSLFFTVLQDVPVRISEQID NO: 25);
LGNWSAMPSCK (SEQ ID NP: 26); and
QVFPGLNYCTSGAYSNASSTDSASYYPLTGD (SEQ ID NO: 27).

60. (canceled)
